SEQUENCE LISTING (3 sequences)

SEQ ID NO: 1 - DNA, 1002 bp, Artificial Sequence
  nucleotide polymorphism
attagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatctgtgagggcagcaaaggaggccaacatacatgccttcatcgagtcactgcctaatgtaagtctctcttcaaataaacagcctgggagcatgtggcagcctctctggcctatagtttgatttataaggggctggtctcccagaagtgaagagaaattagcaaccaaatcacacccttacctgtatacaagcatctggccacacttcctgtttgggttagttgttacctttacctgatcacctgaccctccttgtgaggaagggatgaaagtgttcgaccacttcaggtttaggagagaggaacatttctgggataggagaactggaacaattgtcttgatccaaagctataggcttgaggctccacctttgtcagccttaggggtaagtacaatatctggaaagcctttcactttaagtccaagtacagagtctgggtccccacctgcacatgctgcttctggcctgctgaggaagtaggcatgactgtctctccccatgtc

SEQ ID NO: 2 - DNA, 41 bp, Artificial Sequence
  nucleotide polymorphism
gtggtgtcacaggaagagatcgtgagggcagcaaaggaggc

SEQ ID NO: 3 - DNA, 41 bp, Artificial Sequence
  nucleotide polymorphism
gtggtgtcacaggaagagattgtgagggcagcaaaggaggc

(19) United States
(12) Patent Application Publication
(10) Pub. No.: US 2014/0221372 A1
(43) Pub. Date: Aug. 7, 2014

(54) METHOD OF ADMINISTRATION AND TREATMENT

(71) Applicant: GlaxoSmithKline Intellectual Property (NO 2.) Limited, Brentford (GB)

(72) Inventors: Diptee Amit KULKARNI, King of Prussia, PA (US); Colin F Spraggs, Stevenage (GB)

(73) Assignee: GlaxoSmithKline Intellectual Property (NO 2.) Limited, Brentford (GB)

(21) Appl. No.: 14/173,973

(22) Filed: Feb. 6, 2014

Related U.S. Application Data
(60) Provisional application No. 61/761,422, filed on Feb. 6, 2013.

(51) Int. Cl. A61K 31/5377

(52) U.S. Cl. CPC A61K 31/5377
USPC 514/235.2

(57) ABSTRACT

The present invention is directed to methods of administering foretinib or pharmaceutically acceptable salts or solvates thereof a c-Met inhibitor to a patient in need thereof comprising:
determining whether said patient has less than 2 copies of the CAT haplotype in NR1I3 gene and/or the TT or CT genotype at the rs1045642 reference single nucleotide polymorphism in ABCB1 gene; and
if said patient has less than 2 copies of the CAT haplotype in NR1I3 gene and/or the TT or CT genotype at the rs1045642 reference single nucleotide polymorphism in ABCB1 gene, administering to said patient a pharmaceutical composition comprising foretinib or a pharmaceutically acceptable salt thereof.

### FIELD OF THE INVENTION

The present invention relates to the administration of drug and its effects on patients with particular genetic variants.

### BACKGROUND OF THE INVENTION

Hepatocellular carcinoma (HCC) is the sixth and eleventh most common cancer worldwide in men and women, respectively (Hussain, et al. Ann Oncol. 2001; 12:161-72). Globally, over 600,000 new cases are diagnosed each year, and it is the third leading cause of cancer mortality. The geographic areas at highest risk, with age-adjusted incidence rates of greater than 20 per 100,000, are China and eastern Asia, middle Africa, and some countries of western Africa. Moderately high incidences (10 to 20 per 100,000) are found in Japan, southern Europe, Switzerland, and Bulgaria, whereas the lowest risk areas include northern Europe, Australia, New Zealand, and in the Caucasian population in North and Latin America (Lopez J B. Clin Biochem Rev. 2005; 26:65-9).

The majority of HCC cases occur in males, although the male-to-female ratio is far more striking in African and Asian patients (4:1 to 8:1) than in patients in low-incidence regions (2:1 to 3:1). Populations with an intermediate risk of HCC generally have a ratio of about 4:1. The difference in incidence by gender is thought to be contributed to by variations in hepatitis carrier states, differential exposure to environmental toxins, and the trophic effect of androgens (Okuda K. Epidemiology of primary liver cancer. In: Tobe T, editor. Primary liver cancer in Japan. Tokyo: Springer-Verlag; 1992:3).

Known risk factors for HCC include the hepatitis B carrier state, chronic hepatitis C infection, environmental toxins (e.g., aflatoxin), hereditary hemochromatosis, acute and chronic hepatic porphyria, and cirrhosis from any cause (most commonly alcohol). Hepatocellular carcinoma occurs most often in patients over 40 years of age (Lopez supra 2005), consistent with the association of HCC with long-standing liver disease. The prognosis of HCC is generally grave due to local progression and/or metastasis. In the Chinese and African populations, the mean survival time may be as short as 11 weeks from the onset of symptoms and 6 weeks from the time of diagnosis. Comparatively, the disease progresses somewhat more slowly in patients in low-risk regions, although even they have a mean survival of only about 6 months (Lopez supra, 2005). The degree of hepatic dysfunction produced by the various causes of HCC is the most likely reason for the difference in biologic aggressiveness of the disease. Most patients with HCC also suffer from liver cirrhosis; thus, the malignant disease as well as the treatment may jeopardize the fragile balance of liver function.

The receptor for hepatocyte growth factor (HGF), known as mesenchymal epithelial transition factor (c-Met), is a receptor tyrosine kinase (RTK) widely expressed in epithelial and endothelial cells. Its cognate ligand, HGF, is expressed by cells of the mesenchymal lineage, facilitating rigorous regulation of c-Met kinase activity. The signaling of Hgf/c-MET genes is involved in hepatic development and biology in several ways (Michalopoulos G K, DeFrances M C. Liver regeneration. Science. 1997; 276:60-6). Hepatocyte growth factor is a potent mitogen for hepatocytes. Its proliferative effect on hepatocytes and HCC cells is mediated through c-Met. Increased HGF levels after partial hepatectomy promote liver regeneration by enhancing proliferation of mature hepatocytes and hepatic progenitor cells. In patients with HCC, high peripheral and portal HGF serum levels are associated with poor prognosis after hepatic resection (Chau, et al. Eur J Surg Oncol. 2008:34:333-8). In Hgf knockout mice, the hepatic plate is underdeveloped (Schmidt, Nature. 1995; 373:699-702). In adult rat livers, c-Met activation has been shown to alleviate chemically induced fibrosis [Ueki, Nat Med. 1999; 5:226-30] and to protect hepatocytes from CD95-mediated apoptosis. c-Met is a central mediator of cell growth, survival, motility, and morphogenesis during early development. However, its natural role in adults appears to be primarily confined to repair/regeneration following injury of tissues such as liver (Birchmeier, et al. Nat Rev Mol Cell Biol. 2003; 4:915-25). Hepatocyte growth factor and c-Met may thus be effective targets for therapy in HCC.

The c-Met receptor has been implicated as a mediator in many important aspects of tumor pathobiology including tumor survival, growth, angiogenesis, invasion, and dissemination (Birchmeier, et al. Nat Rev Mol Cell Biol. 2003; 4:915-25; Ma, et al. Cancer Res. 2003a; 63:6272-81). The vascular endothelial growth factor (VEGF) receptor VEGFR2 (kinase insert domain receptor [KDR]) is also a central mediator of tumor angiogenesis. In addition to their individual roles in tumor pathobiology, preclinical data suggest that c-Met and VEGFR2/KDR play synergistic roles in promoting tumor angiogenesis and subsequent dissemination (Bottarro and Liotta, Nature. 2003; 423:593-5). Sorafenib (Nexavar, Bayer), a tyrosine kinase inhibitor that inhibits VEGFRs and BRAF (v-raf murine sarcoma viral oncogene homolog B1), has been shown to prolong stable disease (SD) (albeit with minimal tumor shrinkage) (Abou-Alfa, et al. J Clin Oncol. 2006; 24:4293-300). This activity has translated into survival advantage compared with placebo in the first-line treatment of HCC (Llovet, et al. J Clin Oncol 2007 ASCO Annual Meeting Proceedings. 2007; 25(20 June Suppl):LBA1). Preclinical data support a central role for c-Met in tumor pathobiology. The proto-oncogene c-MET regulates metastasis formation, tumor invasion, and angiogenesis [Ma, et al. Cancer Metastasis Rev. 2003b; 22:309-25]. Amplification, activating mutations, and overexpression of c-Met have been associated with poor prognosis and metastatic phenotype in a variety of human cancers such as papillary renal cell carcinoma (PRC) and gastric cancer [Ma, et al. Cancer Metastasis Rev. 2003b; 22:309-25).

Dysregulation of c-Met signaling results in enhanced tumorigenicity and metastatic potential in engineered cells and in transgenic mice (reviewed in Birchmeier, 2003 supra; Ma, 2003a supra). Conversely, inhibition of c-Met expression by use of ribozymes or antisense RNA inhibits growth of diverse human tumor xenografts in mice (Abounader, et al, FASEB J. 2002; 16:108-10; Kim, et al. Clin Cancer Res. 2003; 9:5161-70; Stabile, et al. Gene Ther. 2004; 11:325-35). Additionally, neutralizing antibodies to HGF inhibit the growth of human glioblastoma xenografts in mice, and administration of NK4, the novel antagonistic variant of HGF, inhibits orthotropic growth, invasion, and metastasis of human pancreatic carcinoma cells in mice (Cao, Proc Natl Acad Sci USA. 2001; 98:7443-8; Tomioka, Cancer Res. 2001; 61:7518-24).

Activation and/or overexpression of c-Met have been widely documented as frequent events in all major human tumor types (reviewed in Birchmeier, et al. 2003; Ma, et al. 2003a). In human HCC, overexpression and mutation of the c-MET gene are associated with intrahepatic metastases and vascular invasion (Corso, et al. Trends Mol Med. 2005; 11:284-92). Expression of c-Met has been consistently correlated with more aggressive disease and poor prognosis (D'Errico, et al. Hepatology. 1996; 24:60-4; Daveau, et al. Mol Carcinog. 2003; 36:130-41; Ueki, et al. Hepatology. 1997; 25:862-6). Moreover, inhibition of c-Met by several therapeutic strategies including tyrosine kinase inhibitors [Wang, et al. J Hepatol. 2004; 41:267-73), small interfering RNAs (Zhang, et al. Mol Cancer Ther. 2005; 4:1577-84), and gene therapy (Heideman, et al. Cancer Gene Ther. 2005; 12:954-62) have shown promise in the treatment of in vitro and in vivo preclinical models of HCC. These data validate c-Met as a therapeutic target in HCC. There are few specific c-Met inhibitors under clinical development, and none have so far been tested in the setting of HCC. Another RTK, the receptors for angiopoietin-1 and -2, has also been associated with HCC initiation and progression (Zhang, et al. World J Gastroenterol. 2006; 12:4241-5), and multiple anti-RTK therapeutic strategies have shown promise in the treatment of HCC in preclinical models. c-Met is thus regarded as a promising molecular target for antimetastatic therapies (Chen, et al. Hepatology. 1997; 26:59-66).

Foretinib (also referred to as Formula I herein) is an oral multikinase inhibitor targeting c-Met, Tie-2, RON, Axl, and VEGFR. HGF/Met signaling plays a pivotal role in tumor cell proliferation, migration and invasion, and circulating levels of HGF correlate with poor prognosis in HCC. Compounds that simultaneously inhibit VEGF and c-Met RTKs may be more effective anticancer agents than agents targeting each of these receptors individually (Pennacchietti, et al. Cancer Cell. 2003; 3:347-61. 2003). In addition, foretinib has activity against other RTKs that have been implicated in tumor pathobiology, including the transmembrane tyrosine kinase KIT, platelet-derived growth factor receptors, FMS-like tyrosine kinase 3, and the receptor for angiopoietin-2, Tie-2.

It would be useful to provide novel methods of treatment for an individual suffering from cancer wherein said patient had a longer time to disease progression also referred to herein as time to progression (TTP).

### SUMMARY OF THE INVENTION

This invention provides that patients who have 2 copies of the CAT haplotype (a combination of C, A, and T alleles of the reference single nucleotide polymorphisms (SNPs)—rs2307424, rs2307418, and rs4073054, respectively) in NR1I3 gene or CC genotype at the rs1045642 locus in ABCB1 gene and are administered a c-Met inhibitor to treat a condition such as cancer experience a shorter time to progression (TTP) than patients who have 0 or 1 copy of the CAT haplotype in NR1I3 and/or a TT or CT genotype at the rs1045642 reference single nucleotide polymorphism in ABCB1. According to an aspect of the present invention, are methods of administering a c-Met inhibitor to a patient in need thereof comprising determining whether said patient has less than 2 copies of the CAT haplotype in NR1I3 gene and/or the TT or CT genotype at the rs1045642 SNP locus in ABCB1, and if said patient has less than 2 copies of the CAT haplotype in NR1I3 and/or the TT or CT genotype at the rs1045642 SNP locus in ABCB1, administering to said patient a c-Met inhibitor.

According to another aspect of the present invention are methods of prescribing a c-Met inhibitor to a patient in need thereof comprising determining whether said patient has less than 2 copies of the CAT haplotype in NR1I3 gene and/or the TT or CT genotype at the rs1045642 SNP locus in ABCB1 gene, and if said patient has less than 2 copies of the CAT haplotype in NR1I3 and/or the TT or CT genotype at the rs1045642 SNP locus in ABCB1 gene, prescribing to said patient the c-Met inhibitor.

According to still another aspect of the present invention are methods of treating cancer in a patient in need thereof comprising determining whether said patient has less than 2 copies of the CAT haplotype in NR1I3 gene and/or the TT or CT genotype at the rs1045642 SNP locus in ABCB1 gene, and if said patient has less than 2 copies of the CAT haplotype in NR1I3 gene and/or the TT or CT genotype at the rs1045642 SNP locus in ABCB1 gene, administering to said patient a c-Met inhibitor.

According to another aspect of the present invention are methods of treating cancer in a patient in need thereof, the patient having been previously genotyped as having less than 2 copies of the CAT haplotype in NR1I3 gene and/or a TT or CT genotype at the rs1045642 SNP locus in ABCB1 gene, comprising administering to the patient a c-Met inhibitor.

According to yet another aspect of the present invention are methods of treating cancer in a patient in need thereof comprising administering to the patient a c-Met inhibitor, and then determining whether said patient has less than 2 copies of the CAT haplotype in NR1I3 gene and/or the TT or CT genotype at the rs1045642 SNP locus in ABCB1 gene.

The present invention also provides methods treating cancer in a patient in need thereof comprising:

administering to the patient a c-Met inhibitor; and then

determining whether said patient has less than 2 copies of the CAT haplotype in NR1I3 gene and/or the TT or CT genotype at the rs1045642 SNP locus in ABCB1 gene.

The present invention also provides methods of treating a human with cancer comprising:
determining whether said human has less than 2 copies of the CAT haplotype in NR1I3 gene; and
determining whether said human has the TT or CT genotype at the rs1045642 SNP locus in ABCB1 gene; and

if said human has the TT or CT genotype at the rs1045642 SNP locus in ABCB1 gene and/or has less than 2 copies of the CAT haplotype in the NR1I3 gene, administering to said human a c-Met inhibitor.

### BRIEF DESCRIPTION OF THE DRAWINGS

FIG. 1: Association of ABCB1 rs1045642 (3435 C>T) CC vs CT or TT (p=0.11) genotypes and time to progression in patients treated for hepatocellular cancer (HCC) with foretinib. CC carriers (n=8) had median TTP of −3 months compared to 8 months in patients carrying TT or CT (n=22) genotypes (p=0.11).

FIG. 2: Association CAT Haplotype (CAT/CAT vs. −/CAT and −/−; p=0.024) in NR1I3 with time to progression in patients treated for hepatocellular cancer (HCC) with foretinib. Carriers of 2 copies of the CAT haplotype (n=4) had median TTP of ˜2 months compared to 6 months in patients carrying other haplotypes (n=26) of NR1I3.

FIG. 3: Effect of IL6 on TTP using median split of IL6 expression.

FIG. 4: Effect of levels of IL6 on OS, depicted in quartiles, and effect of levels of IL8 on OS, depicted in quartiles. (Q1-Q4=1^{st} through 4^{th} quartiles.)

FIG. 5: Correlation between IL6 and IL8.

FIG. 6: CAF levels for IL6 and IL8: prediction of survival.

FIG. 7: Optimal combination of CAF levels for IL6 and IL8.

### DETAILED DESCRIPTION OF THE INVENTION

As used herein, the term “solvate” refers to a complex of variable stoichiometry formed by a solute (in this invention, compounds of formula (I), or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. In particular embodiments, the solvent used is water. One of ordinary skill in the art will readily appreciate how to determine if a solvate of compounds I, I′, and/or I″ will form and how to determine the composition of the solvate using standard solvate screening technology understood by those skilled in the art, for example.

rs1045642, also known as C3435T, is a SNP located in the ABCB1 (ATP-binding cassette, sub-family B) (MDR/TAP), member 1gene (Gene ID: 5243). While the rs1045642 reference single nucleotide polymorphism is understood to have the sequence:

(SEQ ID NO: 1)
ATTAGCAACC TTACATCTAC TACTTTAGTT TCTTTTTGCC
ATGTAACATA ACACATTCAC AGGATCCAGG GATTAGGACA
CAGATGTCTT GTGGGAGAGG GAACATTATT CTGCCTACCA
CATGCATACA TCAGAAACCA TGGTTGAAAC ACAGGAAACA
TGACAGTTCC TCAAGGCATA CAATTATGAC CTTGTTGGGT
TAACCTTCAC TATCCAAATT TTAATCACAC AAACTTTTCC
TTAATCTCAC AGTAACTTGG CAGTTTCAGT GTAAGAAATA
ATGATGTTAA TTGTGCTACA TTCAAAGTGT GCTGGTCCTG
AAGTTGATCT GTGAACTCTT GTTTTCAGCT GCTTGATGGC
AAAGAAATAA AGCGACTGAA TGTTCAGTGG CTCCGAGCAC
ACCTGGGCAT CGTGTCCCAG GAGCCCATCC TGTTTGACTG
CAGCATTGCT GAGAACATTG CCTATGGAGA CAACAGCCGG
GTGGTGTCAC AGGAAGAGAT
C/T
GTGAGGGCAG CAAAGGAGGC CAACATACAT GCCTTCATCG
AGTCACTGCC TAATGTAAGT CTCTCTTCAA ATAAACAGCC
TGGGAGCATG TGGCAGCCTC TCTGGCCTAT AGTTTGATTT
ATAAGGGGCT GGTCTCCCAG AAGTGAAGAG AAATTAGCAA
CCAAATCACA CCCTTACCTG TATACAAGCA TCTGGCCACA
CTTCCTGTTT GGGTTAGTTG TTACCTTTAC CTGATCACCT
GACCCTCCTT GTGAGGAAGG GATGAAAGTG TTCGACCACT
TCAGGTTTAG GAGAGAGGAA CATTTCTGGG ATAGGAGAAC
TGGAACAATT GTCTTGATCC AAAGCTATAG GCTTGAGGCT
CCACCTTTGT CAGCCTTAGG GGTAAGTACA ATATCTGGAA
AGCCTTTCAC TTTAAGTCCA AGTACAGAGT CTGGGTCCCC
ACCTGCACAT GCTGCTTCTG GCCTGCTGAG GAAGTAGGCA
TGACTGTCTC TCCCCATGTC

### Polymorphism Description:

This is a C/T SNP in the coding region of ABCB1 gene. This SNP results in synonymous substitution Isoleucine (ATT)->Isoleucine (ATC) at amino acid position 1145 of the gene.

### Alleles:

| Allele | Allele | |
|---|---|---|
| Name | Symbol | Description |
| C(Ile) | C | 5′-gtggtgtcacaggaagag AT [symbol] |
| | | gtgagggcagcaaaggaggc-3′ |
| | | (SEQ ID NO: 2) |
| T(Ile) | T | 5′-gtggtgtcacaggaagag AT [symbol] |
| | | gtgagggcagcaaaggaggc-3′ |
| | | (SEQ ID NO: 3) |

Those skilled in the art will appreciate that polymorphisms which are similar to the [C/T] polymorphism shown in the sequence can also exist, namely [C/G] and [C/A].

The rs1045642 reference single nucleotide polymorphisms for which a sequence is shown above can be detected using various oligonucleotides designed around this sequence as will be understood by those skilled in the art.

NR1I3 (CAR-Constitutive Androstane Receptor) regulates the activity of CYP3A4-CYP primarily responsible for foretinib metabolism. NR1I3 gene is highly polymorphic and has numerous splice variants. The CAT haplotype is described below.

SNPs in the haplotype:
rs2307424: 5719C/T; synonymous SNP; P180P
rs2307418: 7738A/C; intronic SNP
rs4073054: 7837T/G; intronic SNP
See http://www.ncbi.nlm.nih.gov/sites/entrez for various polymorphisms.

The reference for the primary source for this gene is Hugo Gene Nomenclature Committee (HGNC): 7969. This gene encodes a member of the nuclear receptor superfamily, and is a key regulator of xenobiotic and endobiotic metabolism. The protein binds to DNA as a monomer or a heterodimer with the retinoid X receptor and regulates the transcription of target genes involved in drug metabolism and bilirubin clearance, such as cytochrome P450 family members. Unlike most nuclear receptors, this transcriptional regulator is constitutively active in the absence of ligand but is regulated by both agonists and inverse agonists. Ligand binding results in translocation of this protein to the nucleus, where it activates or represses target gene transcription. These ligands include bilirubin, a variety of foreign compounds, steroid hormones, and prescription drugs. Multiple transcript variants encoding different isoforms have been found for this gene.

The term “wild type” as is understood in the art refers to a polypeptide or polynucleotide sequence that occurs in a native population without genetic modification. As is also understood in the art, a “variant” includes a polypeptide or polynucleotide sequence having at least one modification to an amino acid or nucleic acid compared to the corresponding amino acid or nucleic acid found in a wild type polypeptide or polynucleotide, respectively. Included in the term variant is Single Nucleotide Polymorphism (SNP) where a single base pair distinction exists in the sequence of a nucleic acid strand compared to the most prevalently found (wild type) nucleic acid strand. As used herein “genetic modification” or “genetically modified” refers to, but is not limited to, any suppression, substitution, deletion and/or insertion of one or more bases into DNA sequence(s). Also, as used herein “genetically modified” can refer to a gene encoding a polypeptide or a polypeptide having at least one deletion, substitution or suppression of a nucleic acid or amino acid, respectively.

As used herein, a “gene” is a sequence of DNA present in a cell that directs the expression of a “gene product,” most commonly by transcription to produce RNA and translation to produce protein. An “allele” is a particular form of a gene. The term allele is relevant when there are two or more forms of a particular gene. Genes and alleles are not limited to the open reading frame of the genomic sequence or the cDNA sequence corresponding to processed RNA. A gene and allele can also include sequences upstream and downstream of the genomic sequence such as promoters and enhancers. The term “gene product” or “polymorphic variant allele product” refer to a product resulting from transcription of a gene. Gene and polymorphic variant allele products include partial, precursor, mature transcription products such as pre-mRNA and mRNA, and translation products with or without further processing including, without limitation, lipidation, phosphorylation, glycosylation, other modifications known in the art, and combinations of such processing. RNA may be modified without limitation by complexing with proteins, polyadenylation, splicing, capping or export from the nucleus.

A “polymorphism” is a site in the genome that varies between two or more individuals or within an individual in the case of a heterozygote. The frequency of the variation can be defined above a specific value for inclusion of variations generally observed in a population as opposed to random mutations. Polymorphisms that can be screened according to the invention include variation both inside and outside the open reading frame. When outside the reading frame the polymorphism can occur within 200, 500, 1000, 2000, 3000, 5000, or more of either the 5′ or 3′ end of the open reading frame. When inside the reading frame, the polymorphism may occur within an exon or intron, or overlapping an exon/intron boundary. A polymorphism could also overlap the open reading frame and a sequence outside of that frame. Many polymorphisms have been given a “rs” designation in the SNP database of NCBI's Entrez, some of these designations have been provided herein.

A “polymorphic variant” is a particular form or embodiment of a polymorphism. For example, if the polymorphism is a single nucleotide polymorphism, a particular variant could potentially be an “A” (adenosine), “G” (guanine), “T” (thymine), and “C” (cytosine). When the variant is a “T”, it is understood that a “U” (uracil) can occur in those instances wherein the relevant nucleic acid molecule is RNA, and “C” (cytosine) in respect to DNA. The convention “PositionNUC1>NUC2” is used to indicate a polymorphism contrasting one variant from another. For example, 242A>C would refer to a cytosine instead of an adenosine occurring at position 242 of a particular nucleic acid sequence. In some cases, the variation can be to two or more different bases, e.g., 242A>C/T. When 242A>C is used in respect to a mRNA/cDNA, it can also be used to represent the polymorphism as it occurs in the genomic DNA with the understanding that the position number will likely be different in the genome. Sequence and polymorphic location information for both coding domain sequence and genomic sequence is described herein for the genes relevant to the invention. “Polymorphic variant allele” refers to an allele comprising a particular polymeric variant or a particular set of polymorphic variants corresponding to a particular set of polymorphisms. Two alleles can both be considered the same polymorphic variant allele if they share the same variant or set of variants defined by the polymorphic variant allele even though they may differ in respect to other polymorphisms or variation outside the definition. For a mutation at the amino acid level, the convention “AA1 PositionAA2” is used. For example, in the context of amino acid sequence, M726L, would indicate that the underlying, nucleotide level polymorphism(s) has resulted in a change from a methionine to a leucine at position 726 in the amino acid sequence.

A “genotype” can refer to a characterization of an individual's genome in respect to one or both alleles and/or one or more polymorphic variants within that allele. A subject can be characterized at the level that the subject contains a particular allele, or at the level of identifying both members of an allelic pair, the corresponding alleles on the set of two chromosomes. One can also be characterized at the level of having one or more polymorphic variants. The term “haplotype” refers to a cis arrangement of two or more polymorphic variants, on a particular chromosome such as in a particular gene. The haplotype preserves the information of the phase of the polymorphic nucleotides—that is, which set of polymorphic variants were inherited from one parent, and which from the other. Wherein methods, materials, and experiments are described for the invention in respect to polymorphic variants, one will understand that can also be adapted for use with an analogous haplotype. A “diplotype” is a haplotype that includes two polymorphisms.

A single nucleotide polymorphism (SNPs) refers to a variation at a single nucleotide location. In some cases the variations at the position could be any one of the four nucleotide bases, in others the variation is some subset of the four bases. For example, the variation could be between either purine base or either pyrimidine base. Simple-sequence length polymophisms (SSLPs) or short tandem repeat polymorphisms (STRPs) involve the repeat of a particular sequence of one or more nucleotides. A restriction fragment length polymorphism (RFLP) is a variation in the genetic sequence that results in the appearance or disappearance of an enzymatic cleavage site depending on which base(s) are present in a particular allele.

Genetic variants and/or SNPs can be identified by known methods. For example, wild type or SNPs can be identified by DNA amplification and sequencing techniques, DNA and RNA detection techniques, including, but not limited to Northern and Southern blot, respectively, and/or various biochip and array technologies. WT and mutant polypeptides can be detected by a variety of techniques including, but not limited to immunodiagnostic techniques such as ELISA and western Blot.

As used herein, the process of detecting an allele or polymorphism includes but is not limited to serologic and genetic methods. The allele or polymorphism detected may be functionally involved in affecting an individual's phenotype, or it may be an allele or polymorphism that is in linkage disequilibrium with a functional polymorphism/allele. Polymorphisms/alleles are evidenced in the genomic DNA of a subject, but may also be detectable from RNA, cDNA or protein sequences transcribed or translated from this region, as will be apparent to one skilled in the art.

As is well known in the study of genetics, nucleotide and related amino acid sequences obtained from different sources for the same gene may vary both in the numbering scheme and in the precise sequence. Such differences may be due to numbering schemes, inherent sequence variability within the gene, and/or to sequencing errors. Accordingly, reference herein to a particular polymorphic site by number will be understood by those of skill in the art to include those polymorphic sites that correspond in sequence and location within the gene, even where different numbering/nomenclature schemes are used to describe them.

As used herein, “genotyping” a subject (or DNA or other biological sample) for a polymorphic allele of a gene(s) means detecting which allelic or polymorphic form(s) of the gene(s) or gene expression products (e.g., hnRNA, mRNA or protein) are present or absent in a subject (or a sample). Related RNA or protein expressed from such gene may also be used to detect polymorphic variation. As is well known in the art, an individual may be heterozygous or homozygous for a particular allele. More than two allelic forms may exist, thus there may be more than three possible genotypes. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g., sequencing) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with foretinib or individuals with cancer.

As used herein, a subject that is “predisposed to” or “at increased risk of” a particular phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies. A commonly used measure of linkage disequilibrium is r:

$$r = \frac{{\hat{Δ}}_{AB}}{\sqrt{( {\tilde{π}}_{A} + {\hat{D}}_{A} ) ( {\tilde{π}}_{B} + {\hat{D}}_{B} )}}$$

$$where$$

$${\tilde{π}}_{A} = {\tilde{p}}_{A} ( 1 - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2}$$

$${\hat{Δ}}_{AB} = \frac{1}{n} {n}_{AB} - 2 {\tilde{p}}_{A} {\tilde{p}}_{B}$$

nr² has an approximate chi square distribution with 1 degree freedom for biallelic markers. Loci exhibiting an r such that nr² is greater than 3.84, corresponding to a significant chi-squared statistic at the 0.05 level, are considered to be in linkage disequilibrium (BS Weir 1996 Genetic Data Analysis II Sinauer Associates, Sunderland, Md.).

Alternatively, a normalized measure of linkage disequilibrium can be defined as:

$${D}_{AB}^{/} = \{ \begin{matrix}\frac{{D}_{AB}}{min ( {p}_{A} {p}_{B} , {p}_{a} {p}_{b} )} , & {D}_{AB} < 0 \\ \frac{{D}_{AB}}{min ( {p}_{A} {p}_{b} , {p}_{a} {p}_{B} )} , & {D}_{AB} > 0\end{matrix}$$

The value of the D′ has a range of −1.0 to 1.0. When statistically significant absolute D′ value for two markers is not less than 0.3 they are considered to be in linkage disequilibrium.

As used herein, “treatment” means any manner in which one or more symptoms associated with the disorder are beneficially altered. Accordingly, the term includes healing or amelioration of a symptom or side effect of the disorder or a decrease in the rate of advancement of the disorder.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell. By way of example, a ras protein may be overexpressed by a tumor cell relative to a non-tumor cell. Additionally, a mutant ras protein may be overexpressed compared to wild type ras protein in a cell. As is understood in the art, expression levels of a polypeptide in a cell can be normalized to a housekeeping gene such as actin. In some instances, a certain polypeptide may be underexpressed in a tumor cell compared with a non-tumor cell.

Foretinib (also referred to herein as N¹-{3-fluoro-4-[(6-(methyloxy)-7-{[3-(4-morpholinyl)propyl]oxy}-4-quinolinyl)oxy]phenyl}-N¹-(4-fluorophenyl)-1,1-cyclopropanedicarboxamide), is disclosed and claimed, along with pharmaceutically acceptable salts and solvates thereof, methods of preparation, and as being useful as an inhibitor of cMET, particularly in treatment of cancer, in International Application No. PCT/US2004/031523, having an International filing date of Sep. 24, 2004; International Publication Number WO2005/030140 and an International Publication date of Apr. 7, 2005, the entire disclosure of which is hereby incorporated by reference. Examples 25 (p. 193), 36 (pp. 202-203), 42 (p. 209), 43 (p. 209), and 44 (pp. 209-210) describe how Formula I can be prepared. Formula I can be prepared as described in International Application No. PCT/US2009/064341 having an International filing date of Nov. 13, 2008; International Publication Number WO2010/056960 and an International Publication date of May 20, 2010, the entire disclosure of which is hereby incorporated by reference and in International Application No. PCT/US2009/058276 having an International filing date of Sep. 25, 2009; International Publication Number WO2010/036831 and an International Publication date of Apr. 1, 2010 the entire disclosure of which is hereby incorporated by reference.

The general preparation for Formula I is outlined in Scheme 1:

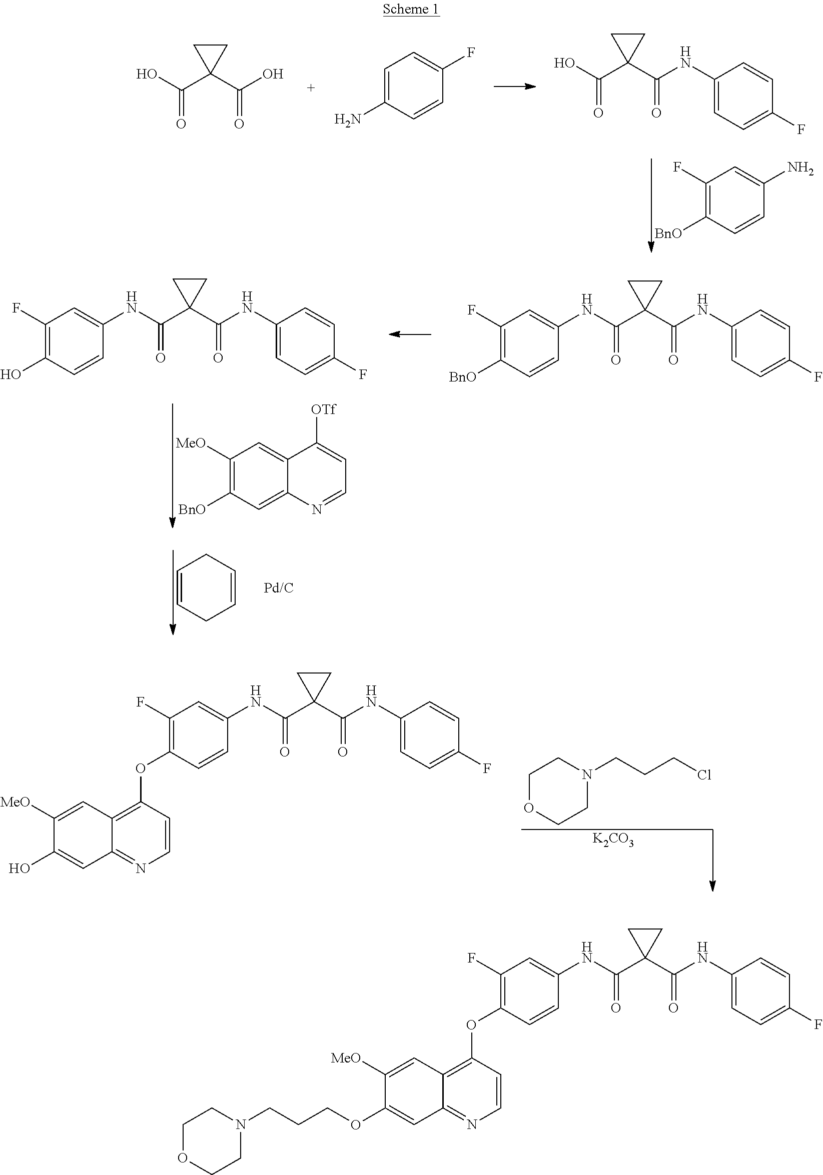

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; to angiogenesis inhibitors, immunotherapeutic agents; proapoptotic agents; and cell cycle signalling inhibitors.

The present invention also provides methods for treating cancer comprising administering Formula I or pharmaceutically acceptable salt thereof with or without another anti-neoplastic agent.

Various c-Met inhibitors can be used in the methods of the present invention. In some embodiments, the c-Met inhibitor is the compound of Formula (I) or a pharmaceutically acceptable salt or solvate thereof:

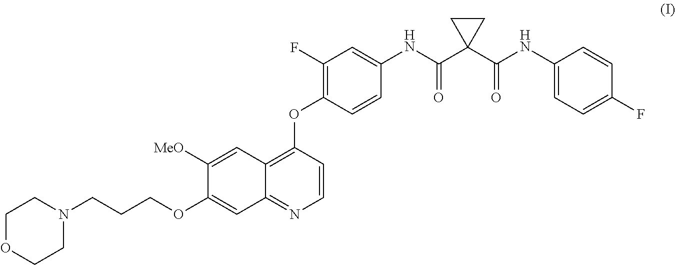 [SMILES: COc1cc2c(Oc3ccc(NC(=O)C4(C(=O)Nc5ccc(F)cc5)CC4)cc3F)ccnc2cc1OCCCN1CCOCC1]

which has the chemical name: N¹-{3-fluoro-4-[(6-(methyloxy)-7-{[3-(4-morpholinyl)propyl]oxy}-4-quinolinyl)oxy]phenyl}-N¹-(4-fluorophenyl)-1,1-cyclopropanedicarboxamide) and is known by the generic name foretinib.

In one aspect, compound comprising Formula I is administered as a free base. In another aspect a compound comprising Formula I may be a pharmaceutically acceptable salt thereof, including but not limited to, a bisphosphate salt form. Formula I can be administered at a dose of at least 7.5 mg daily. Formula I can be administered, for instance, at a dose of about 7.5 mg, 15.0 mg, 30.0 mg and/or 45.0 mg daily. Formula I may be provided in tablet form. In some instances, tablets comprise hypromellose, sodium lauryl sulfate, lactose monohydrate, microcrystalline cellulose, croscarmellose sodium, and magnesium stearate. Some tablets may comprise hypromellose, titanium dioxide, polyethylene glycol. Tablets may comprise polysorbate 80 and iron oxide yellow.

As used herein, the term “pharmaceutically acceptable salts” may comprise acid addition salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, bisphosphate, polygalacturonate, potassium, salicylate, sodium, stearate, subacetate, succinate, tannate, tartrate, teoclate, tosylate, triethiodide, trimethylammonium and valerate.

In other embodiments, the c-Met inhibitors are multikinase inhibitors including but not limited to sunitinib, sorafenib, and axitinib.

In embodiments according to the various aspects of the present invention described herein, the determination of whether a patient has a particular genotype at a given reference single nucleotide polymorphism includes testing the patient for the particular genotype at the given reference single nucleotide polymorphism. The testing of a patient to determine whether the patient has a particular genotype at a given reference single nucleotide polymorphism can be done by various methods as will be understood by those skilled in the art, for example as described in the Examples section below.

In embodiments according to the various aspects of the present invention described herein, the determination of whether a patient has a particular genotype at a given reference single nucleotide polymorphism includes testing the patient for at least one single nucleotide polymorphism that is correlated with the given reference single nucleotide polymorphism.

As used herein, a first reference single nucleotide polymorphism is correlated to a second single nucleotide polymorphism if detection of the first reference single nucleotide polymorphism, or a particular genotype of the first single nucleotide polymorphism, indicates that the individual would have the second reference single nucleotide polymorphism, or a particular genotype of the second reference single nuclear polymorphism, if the individual were to be tested for the second reference single nucleotide polymorphism or particular genotype thereof.

In some embodiments according to the present invention, the determination of whether a patient has a particular genotype at a given reference single nucleotide polymorphism includes:
a. performing a genotyping technique on a biological sample from the subject to determine whether the subject has a TT or CT genotype at the rs1045642 reference single nucleotide polymorphism in ABCB1 gene and/or less than 2 copies of the CAT haplotype at the 3 SNP Loci (rs2307424, rs2307418, and rs4073054) in NR1I3 gene;
b. detecting the TT or CT genotype at the rs1045642 reference single nucleotide polymorphism in ABCB1 gene and/or less than 2 copies of the CAT haplotype at the 3 SNP Loci (rs2307424, rs2307418, and rs4073054) in NR1I3 gene; and
c. correlating the detection of the TT or CT genotype at the rs1045642 reference single nucleotide polymorphism in ABCB1 gene and/or less than 2 copies of the CAT haplotype at the 3 SNP loci (rs2307424, rs2307418, and rs4073054) in NR1I3 gene with increased likelihood of experiencing a longer time to progression (TTP) when administered a c-Met inhibitor compared to the likelihood if no TT or CT genotype at the rs1045642 reference single nucleotide polymorphism in ABCB1 gene and/or 2 copies of the CAT haplotype in NR1I3 gene were detected.

In one aspect, patients who have the TT or CT genotype at the rs1045642 reference single nucleotide polymorphism in ABCB1 gene and/or less than 2 copies of the CAT haplotype at the 3 SNP loci (rs2307424, rs2307418, and rs4073054) in NR1I3 gene will experience a longer time to disease progression (TTP) when administered a c-Met inhibitor that patients who do not have the TT or CT genotype at the rs1045642 reference single nucleotide polymorphism in ABCB1 gene and/or less than 2 copies of the CAT haplotype at the 3 SNP loci (rs2307424, rs2307418, and rs4073054) in NR1I3 gene. In one aspect, the patient has hepatocellular carcinoma. In one aspect, the patient receives 30 mg/day of foretinib. In some embodiments, the biological sample is selected from the group consisting of cells, blood, blood components, urine and saliva.

While it is possible that, the compound of formula (I), as well as pharmaceutically acceptable salts and solvates thereof, as well as the various other c-Met inhibitors described herein, may be administered as the raw chemical, it is also possible to present the active ingredient as a pharmaceutical composition. Accordingly, embodiments of the invention further provide pharmaceutical compositions, which include therapeutically effective amounts of the c-Met inhibitor, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing the c-Met inhibitor with one or more pharmaceutically acceptable carriers, diluents or excipients.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additives such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

Dosage unit forms can also be in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

C-Met inhibitors can also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Eye-drop formulations are described further herein below.

Suitable routes for ocular administration include extraocular and intraocular (including, for example, intravitreal, subretinal, subscleral, intrachoroidal, and subconjuctival). It will be appreciated that the preferred route may vary with, for example, the condition of the recipient.

For treatments of the eye, the pharmaceutical formulations may also be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

In some embodiments of the present invention, the pharmaceutical formulations are adapted for intraocular administration by means of intraocular injection or other device for ocular delivery. Examples of ocular devices that may be used in the methods of the invention include periocular or intravitreal devices, contact lenses and liposomes. See, for example, U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; 6,299,895; 6,331,313; 6,416,777; 6,649,184; 6,719,750; 6,660,960; and U.S. Patent Publication Nos. 2003/0064088, 2004/0247645, and, 2005/0113806; each of which is herein incorporated by reference for purposes of their teachings of optical devices.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D,L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein is includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of formula (I) or a pharmaceutically acceptable salt or solvate thereof will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) or a salt or solvate thereof for the treatment of a cancerous condition such as those described herein will generally be in the range of 0.1 to 100 mg/kg body weight of recipient (mammal) per day and more usually in the range of 1 to 12 mg/kg body weight per day. Thus, an effective amount of a salt or solvate thereof can typically be from a lower limit of 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 mg to an upper limit of about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895 or 900 mg of a compound of the formula (I) depending on the condition being treated, the route of administration and the age, weight and condition of the patient. This amount may be given in a single dose per day or in a number (such as two, three, four, five or six) of sub-doses per day such that the total daily dose is the same. An effective amount of a salt or solvate thereof may be determined as a proportion of the effective amount of the compound of formula (I) per se.

The c-Met inhibitor may be employed alone or in combination with other therapeutic agents for the treatment of the above-mentioned conditions. In particular, in anti-cancer therapy, combination with other chemotherapeutic, hormonal or antibody agents is envisaged as well as combination with surgical therapy and radiotherapy. Combination therapies according to the present invention thus comprise the administration of a c-Met inhibitor, and the use of at least one other cancer treatment method, including one or more additional c-Met inhibitors. Preferably, combination therapies according to the present invention comprise the administration of the c-Met inhibitor, and at least one other pharmaceutically active agent, preferably an anti-neoplastic agent. The c-Met inhibitor and the other pharmaceutically active agent(s) may be administered together or separately and, when administered separately this may occur simultaneously or sequentially in any order. The amounts of the c-Met inhibitor and the other pharmaceutically active agent(s) and the relative timings of administration will be selected in order to achieve the desired combined therapeutic effect.

The c-Met inhibitor and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent. The administration in combination of a compound of formula (I) or pharmaceutically acceptable salts or solvates thereof with other anti-neoplastic agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compound of the c-Met inhibitor can include the following:

(1) cell cycle specific anti-neoplastic agents including, but not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine; antimetabolites such as allopurinol, fludurabine, methotrexate, cladrabine, cytarabine, mercaptopurine and thioguanine; and camptothecins such as 9-amino camptothecin, irinotecan, CPT-11 and the various optical forms of 7-(4-methylpiperazino-methylene)-10,11-ethylenedioxy-20-camptothecin;

(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumour antibiotics such as doxorubicin, daunomycin, epirubicin, idarubicin, mitomycin-C, dacttinomycin and mithramycin; and platinum coordination complexes such as cisplatin, carboplatin, and oxaliplatin; and

(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamide, bicalutamide, and cyproterone acetate; LHRH agonists and antagagonists such as goserelin acetate and luprolide, testosterone 5α-dihydroreductase inhibitors such as finasteride; metalloproteinase inhibitors such as marimastat; antiprogestogens; urokinase plasminogen activator receptor function inhibitors; cyclooxygenase type 2 (COX-2) inhibitors such as celecoxib; other angiogenic inhibiting agents such as VEGFR inhibitors and TIE-2 inhibitors; growth factor function inhibitors such as inhibitors of the functions of hepatocyte growth factor; erb-B2, erb-B4, epidermal growth factor receptor (EGFr), platelet derived growth factor receptor (PDGFr), vascular endothelial growth factor receptor (VEGFR) other than those described in the present invention, and TIE-2; and other tyrosine kinase inhibitors such as cyclin dependent inhibitors such as CDK2 and CDK4 inhibitors.

The c-Met inhibitor can be used to provide additive or synergistic effects with certain existing cancer chemotherapies and radiation, and/or be used to restore effectiveness of certain existing cancer chemotherapies and radiation.

In some embodiments according to the various aspects of the present invention, the c-Met inhibitor is administered or prescribed in the treatment of disorders mediated by inappropriate c-Met activity.

The inappropriate c-Met activity referred to herein is any c-Met activity that deviates from the normal c-Met activity expected in a particular mammalian subject. Inappropriate c-Met activity may take the form of, for instance, an abnormal increase in activity, or an aberration in the timing and or control of c-Met activity. Such inappropriate activity may result then, for example, from overexpression or mutation of the protein kinase or ligand leading to inappropriate or uncontrolled activation of the receptor. Furthermore, it is also understood that unwanted c-Met activity may reside in an abnormal source, such as a malignancy.

In embodiments according to aspects of the present invention, the disorder is cancer. In some instances, the cancer is hepatocellular cancer. In some embodiments according to the various aspects of the present invention, the cancer is selected from the group consisting of colon cancer, breast cancer, renal cell carcinoma, melanoma, lung cancer including non-small cell lung cancer and adenocarcinoma, gastric cancer, colorectal cancer, neuroendocrine cancer, thyroid cancer, head and neck cancer, brain cancer, cervical cancer, bladder cancer, esophageal cancer, pancreatic cancer, prostate cancer, mesothelioma, liver-hepatobiliary cancer, multiple myeloma, leukemia, thyroid cancer including Hurthle cell, muscle sarcoma (leiomyosarcoma) and bone sarcoma (chonrosarcoma).

A further aspect of the present invention provides the use of a c-Met inhibitor compound of formula (I) or a pharmaceutically acceptable salt or solvate thereof in the preparation of a medicament for the treatment of cancer and malignant tumours.

In one embodiment of the present invention, methods are provided for treating a human having heptocellular carcinoma comprising determining whether a said human has an elevated level of at least one cytokine or angiogenesis factor (CAF) selected from: IL8, IL6, TSP2, MMP9, and IGFBP1 and if said human does not have and elevated level of at least one of said CAF administering a therapeutically effective amount of a c-Met inhibitor to said human. In one embodiment the c-Met inhibitor is a compound of Formula (I):

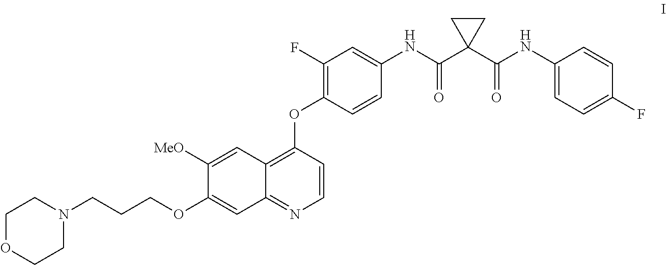 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt thereof.

In one embodiment, patients are of Asian heritage. In one aspect, the c-Met inhibitor is administered at a dose of at least about 30 mg daily. In another aspect, the c-Met inhibitor is administered as monotherapy. In another aspect, the c-Met inhibitor is co-administered with at least one other anti-neoplastic agent.

In another embodiment, the present invention provides a pharmaceutical composition comprising a compound of formula (I):

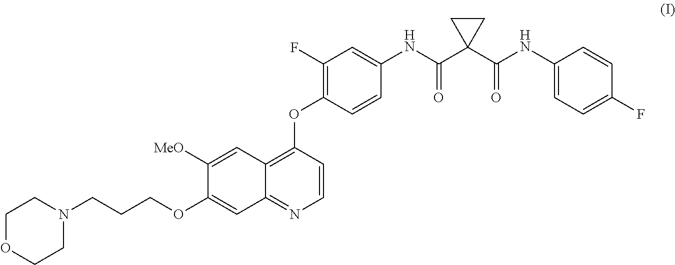 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof for use in the treatment of cancer in a human classified as a responder wherein a responder has less than 2 copies of the CAT haplotype in NR1I3 gene and/or the TT or CT genotype at the rs1045642 SNP locus in ABCB1 gene. In one aspect the cancer is hepatocellular carcinoma.

In another embodiment, kits are provided for detecting the presence of the CAT haplotype in NR1I3 gene and/or the TT or CT genotype at the rs1045642 SNP locus in ABCB1 gene. In another aspect, the kit further comprises a pharmaceutical composition comprising a compound of formula (I):

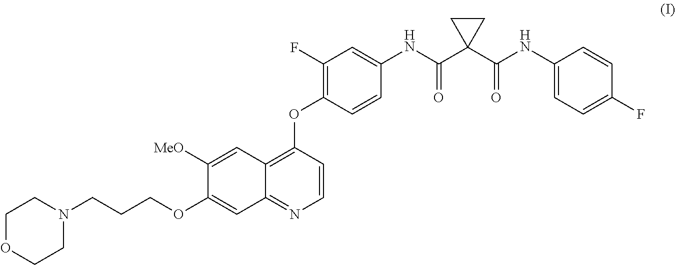 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof.

In one embodiment, the present invention provides the use of a pharmaceutical composition comprising a compound of formula (I):

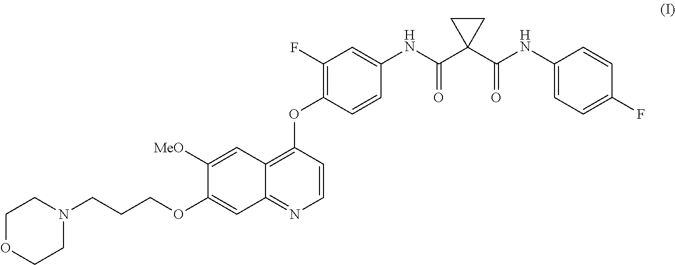 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof for the treatment of HCC in a mammal wherein said mammal has less than 2 copies of the CAT haplotype in NR1I3 gene and/or the TT or CT genotype at the rs1045642 reference single nucleotide polymorphism in ABCB1 gene.

In another embodiment, the present invention provides the use of a pharmaceutical composition comprising a compound of formula (I):

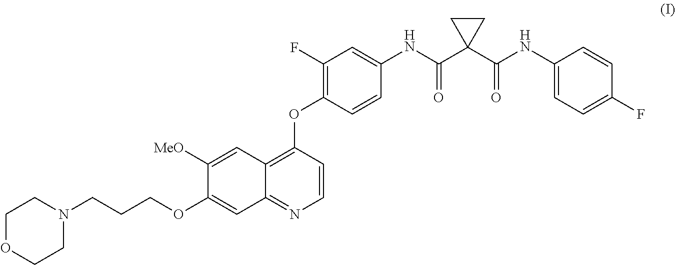 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof in the manufacture of a medicament for the treatment of HCC in a mammal wherein said mammal has less than 2 copies of the CAT haplotype in NR1I3 gene and/or the TT or CT genotype at the rs1045642 reference single nucleotide polymorphism in ABCB1 gene. As is understood in the art mammal includes human patients and subjects.

Where a combination therapy is employed, the therapeutic agents may be administered together or separately. The same means for administration may be used for more than one therapeutic agent of the combination therapy; alternatively, different therapeutic agents of the combination therapy may be administered by different means. When the therapeutic agents are administered separately, they may be administered simultaneously or sequentially in any order, both close and remote in time. The amounts of the compound of formula (I) or a pharmaceutically acceptable salt or solvate thereof and/or the other pharmaceutically active agent or agents and the relative timings of administration will be selected in order to achieve the desired combined therapeutic effect.

The amount of administered or prescribed Formula Iccording to these aspects of the present invention will depend upon a number of factors including, for example, the age and weight of the patient, the precise condition requiring treatment, the severity of the condition, the nature of the formulation, and the route of administration. Ultimately, the amount will be at the discretion of the attendant physician.

Pharmaceutical formulations for administration to the eye may be presented in unit dose forms containing a predetermined amount of active ingredient per unit dose. Such a unit may contain, for example, 1 μg to 1 g, such as 5 μg to 500 μg, 10 μg-250 μg, 0.5 mg to 700 mg, 2 mg to 350 mg, or 5 mg to 100 mg of a compound of formula (I) or pharmaceutically acceptable salts or solvates thereof depending on the condition being treated, the route of administration and the age, weight and condition of the patient, or pharmaceutical formulations may be presented in unit dose forms containing a predetermined amount of active ingredient per unit dose. In some embodiments, the unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art. In some embodiments, the compound of formula (I) or a pharmaceutically acceptable salt or solvate thereof is administered or prescribed to be administered one, two, three, four, or more times per day. In some embodiments, the compound of formula (I) or a pharmaceutically acceptable salt or solvate thereof is administered or prescribed to be administered by administering one, two, three, four or more drops of a suitable pharmaceutical formulation one, two, three, four, or more times per day. In some embodiments of pharmaceutical formulations suitable for topical administration to the eye, the suitable pharmaceutical formulation comprises between a lower limit of 1, 2, 3, 4, 5, 6, 7, 8, or 9 and an upper limit of 2, 3, 4, 5, 6, 7, 8, 9, 10, 11, 12, 13, 14, 15, 16, 17, 18, 19, 20, 30 or 45 mg of a compound of formula (I) or a pharmaceutically acceptable salt or solvate thereof per ml.

In one embodiment, of the present invention, methods are provided comprising administering a c-Met inhibitor to a patient in need thereof comprising:

determining whether said patient has less than 2 copies of the CAT haplotype in NR1I3 gene and/or the TT or CT genotype at the rs1045642 reference single nucleotide polymorphism in ABCB1 gene; and

if said patient has less than 2 copies of the CAT haplotype in NR1I3 gene and/or the TT or CT genotype at the rs1045642 reference single nucleotide polymorphism in ABCB1 gene, administering to said patient the c-Met inhibitor.

In one embodiment of the present invention methods are provided for prescribing a c-Met inhibitor to a patient in need thereof comprising:

determining whether said patient has less than 2 copies of the CAT haplotype in NR1I3 gene and/or the TT or CT genotype at the rs1045642 reference single nucleotide polymorphism in ABCB1 gene; and

if said patient has less than 2 copies of the CAT haplotype in NR1I3 gene and/or TT or CT genotype at the rs1045642 reference single nucleotide polymorphism in ABCB1 gene, prescribing to said patient the c-Met inhibitor.

In one embodiment of the present invention methods are provided for treating cancer in a patient in need thereof comprising:

determining whether said patient has less than 2 copies of the CAT haplotype in NR1I3 gene and/or TT or CT genotype at the rs1045642 reference single nucleotide polymorphism in ABCB1 gene; and

if said patient has less than 2 copies of the CAT haplotype in NR1I3 gene and/or TT or CT genotype at the rs1045642 reference single nucleotide polymorphism in ABCB1 gene, administering to said patient a c-Met inhibitor.

In one aspect, said determining comprises testing said patient for the CAT haplotype in NR1I3 gene and/or TT or CT genotype at the rs1045642 reference single nucleotide polymorphism in ABCB1 gene. In one aspect, said determining comprises testing said patient for the presence of less than 2 copies of the CAT haplotype in NR1I3 gene and/or the TT or CT genotype at the rs1045642 reference single nucleotide polymorphism in ABCB1 gene. In one aspect, said determining comprises genotyping said patient for the presence of less than 2 copies of CAT haplotype in NR1I3 gene and/or TT or CT genotype at the rs1045642 reference single nucleotide polymorphism in ABCB1 gene. In one aspect, the patient in need of treatment has previously been genotyped as having less than 2 copies of the CAT haplotype in NR1I3 gene and/or TT or CT genotype at the rs1045642 reference single nucleotide polymorphism in ABCB1 gene. Several techniques for testing a sample for a SNP and/or genotyping a sample are known in the art and are described herein.

In another embodiment methods are provided of treating cancer in a patient in need thereof comprising:

administering to the patient a c-Met inhibitor; and then

determining whether said patient has less than 2 copies of the CAT haplotype in NR1I3 gene and/or the TT or CT genotype at the rs1045642 reference single nucleotide polymorphism in ABCB1 gene.

In one aspect the cancer is hepatocellular carcinoma. In another aspect, the cancer is selected from the group consisting of: colon cancer, breast cancer, renal cell carcinoma, melanoma, lung cancer including non-small cell lung cancer and adenocarcinoma, gastric cancer, colorectal cancer, neuroendocrine cancer, thyroid cancer, head and neck cancer, brain cancer, cervical cancer, bladder cancer, esophageal cancer, pancreatic cancer, prostate cancer, mesothelioma, liver-hepatobiliary cancer, multiple myeloma, leukemia, thyroid cancer including Hurthle cell, muscle sarcoma (leiomyosarcoma), hepatocellular cancer and bone sarcoma (chonrosarcoma). As is understood in the art, a patient may have more than one type of cancer.

In one aspect the cancer the c-Met inhibitor is a compound of formula (I):

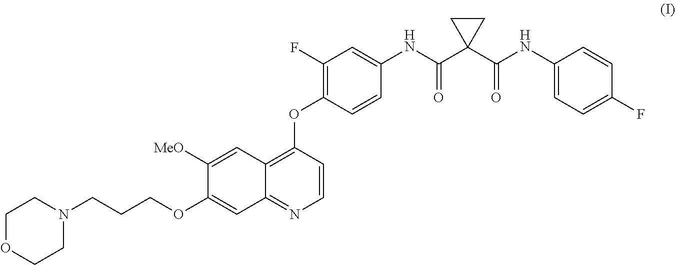 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof. In one aspect, Formula I is the free base form.

In one aspect, the patient has a genotype at at least one single nucleotide polymorphism that is correlated with CT or TT genotype at the rs1045642 reference single nucleotide polymorphism in ABCB1 gene.

In another embodiment, methods are provided further comprising: determining whether said patient has less than 2 copies of CAT haplotype in the NR1I3 gene. NR1I3 (CAR-Constitutive Androstane Receptor) regulates the activity of CYP3A4-CYP primarily responsible for foretinib metabolism. NR1I3 gene is highly polymorphic and has numerous splice variants

SNPs in the haplotype:
rs2307424: 5719C/T; synonymous SNP; P180P
rs2307418: 7738A/C; intronic SNP
rs4073054: 7837T/G; intronic SNP
In one embodiment methods are provided for treating a human with cancer comprising:

determining whether said human has the TT or CT genotype at the rs1045642 reference single nucleotide polymorphism (ABCB1 3435); and/or

determining whether said human has a CAT haplotype in the NR1I3 gene and

if said human has the TT or CT genotype at the rs1045642 reference single nucleotide polymorphism (ABCB1 3435) and/or has less than 2 copies of CAT haplotype in the NR1I3 gene, administering to said human a c-Met inhibitor. In one aspect the c-Met inhibitor is a compound of formula (I):

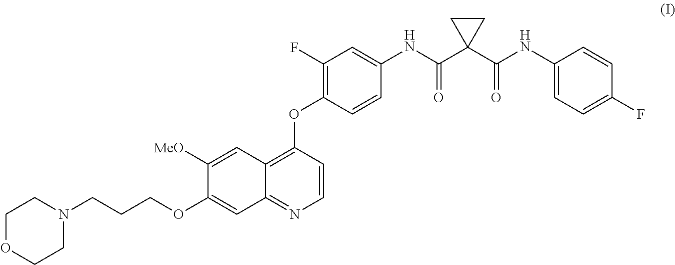 [SMILES: COc1cc2c(Oc3ccc(NC(=O)C4(C(=O)Nc5ccc(F)cc5)CC4)cc3F)ccnc2cc1OCCCN1CCOCC1]

or a pharmaceutically acceptable salt or solvate thereof.

In another embodiment the c-Met inhibitor is co-administered with at least one additional anti-neoplastic agent. Non-limiting examples of other anti-neoplastic agents are described herein.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273,1989) and for the treatment of breast cancer (Holmes et al., J. Nat. Cancer Inst., 83:1797,1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The Formula IIso shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine[1,1-cyclobutane-dicarboxylate(2−)O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-Iyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-Iyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Pazopanib which commercially available as VOTRIENT® is a tyrosine kinase inhibitor (TKI). Pazopanib is presented as the hydrochloride salt, with the chemical name 5-[[4(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

mTOR inhibitors include but are not limited to rapamycin (FK506) and rapalogs, RAD001 or everolimus (Afinitor), CCI-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl) ethenyl]benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino]phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Examples of erbB inhibitors include lapatinib, erlotinib, and gefitinib. Lapatinib, N-(3-chloro-4-{[(3-fluorophenyl)methyl]oxy}phenyl)-6-[5-({[2-(methylsulfonyl)ethyl]amino}methyl)-2-furanyl]-4-quinazolinamine (represented by formula II, as illustrated), is a potent, oral, small-molecule, dual inhibitor of erbB-1 and erbB-2 (EGFR and HER2) tyrosine kinases that is approved in combination with capecitabine for the treatment of HER2-positive metastatic breast cancer.

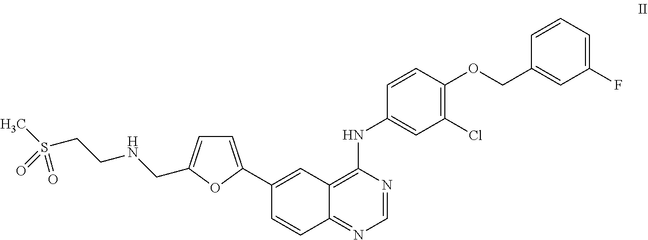 [SMILES: CS(=O)(=O)CCNCc1ccc(-c2ccc3ncnc(Nc4ccc(OCc5cccc(F)c5)c(Cl)c4)c3c2)o1]

The free base, HCl salts, and ditosylate salts of the compound of formula (II) may be prepared according to the procedures disclosed in WO 99/35146, published Jul. 15, 1999; and WO 02/02552 published Jan. 10, 2002.

Erlotinib, N-(3-ethynylphenyl)-6,7-bis{[2-(methyloxy)ethyl]oxy}-4-quinazolinamine (commercially available under the tradename Tarceva) is represented by formula III, as illustrated:

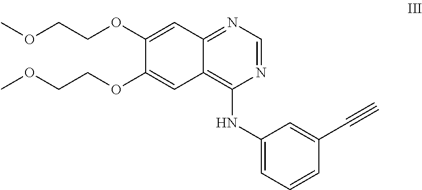 [SMILES: C#Cc1cccc(Nc2ncnc3cc(OCCOC)c(OCCOC)cc23)c1]

The free base and HCl salt of erlotinib may be prepared, for example, according to U.S. Pat. No. 5,747,498, Example 20.

Gefitinib, 4-quinazolinamine,N-(3-chloro-4-fluorophenyl)-7-methoxy-6-[3-4-morpholin)propoxy] is represented by formula IV, as illustrated:

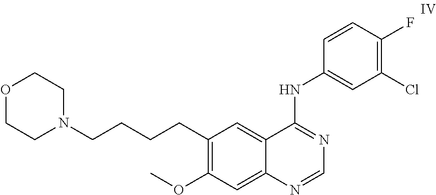 [SMILES: COc1cc2ncnc(Nc3ccc(F)c(Cl)c3)c2cc1CCCCN1CCOCC1]

Gefitinib, which is commercially available under the trade name IRESSA® (Astra-Zenenca) is an erbB-1 inhibitor that is indicated as monotherapy for the treatment of patients with locally advanced or metastatic non-small-cell lung cancer after failure of both platinum-based and docetaxel chemotherapies. The free base, HCl salts, and diHCl salts of gefitinib may be prepared according to the procedures of International Patent Application No. PCT/GB96/00961, filed Apr. 23, 1996, and published as WO 96/33980 on Oct. 31, 1996.

The following examples are intended for illustration only and are not intended to limit the scope of the invention in any way.

### EXAMPLES

### Example I

### Pharmacogenetics Results

### Background

Consistent with other tyrosine kinase inhibitor (TKI) therapies, response to foretinib varies among HCC patients. Biomarkers (serum protein or germline DNA) predictive of efficacy of TKIs such as sorafenib and sunitinib in HCC have previously been reported in small studies (Miyahara 2011, Harmon 2011, and van der Veldt 2011). An exploratory pharmacogenetic (PGx) investigation was conducted to identify germline genetic variants that may explain differences in treatment response in a small Asian population of HCC subjects receiving foretinib.

### Methods:

### Pharmacogenetic Analysis Population:

Asian HCC subjects treated with the maximum tolerated dose (MTD) of 30 mg QD and provided informed consent and blood sample for PGx research (n=31 of 39 subjects treated) were selected for PGx analysis. Of the 31 subjects who provided consent, 28 subjects were evaluable for objective response rate (ORR) and 30 for time to progression (TTP) and overall survival (OS).

### Blood Sampling, DNA Preparation and Genotyping:

Venous blood was collected from each PGx consented subject into an ethylenediaminetetraacetic acid (EDTA) vacutainer for PGx analysis. DNA was extracted using the Qiagen Autopure automated DNA extraction by Covance (Indianapolis, Ind., USA) and was genotyped for 20 single nucleotide polymorphisms (SNPs) with potential functional consequence from 13 candidate genes. The list of the genetic variants including their unique identifier (rs ID) and allele frequency in Asian population is included in Table 1. Genotyping of the SNPs was done via Sanger sequencing, or using custom TaqMan® SNP genotyping assays (Applied Biosystems, Foster City, Calif., USA) internally at GlaxoSmithKline or via TaqMan® custom or Assay on Demand genotyping assays (Applied Biosystems, Foster City, Calif., USA) by Gen-Probe (Wythenshawe, Manchester, UK).

### Pharmacogenetic Analysis Methods:

Association with ORR was evaluated using Fisher's Exact Test (FET). Association with TTP and OS was tested using time-to-event (progression) models, using the Score (log rank) test. Since one of the haplotypes CAT (a unique combination of ‘C’, ‘A’, and ‘T’ alleles of SNPs—rs2307424, rs2307418, and rs4073054, respectively) in gene NR1I3/CAR (nuclear receptor subfamily 1, group I, member 3/constitutive androstane receptor) was previously reported to be associated with worse progression-free survival in sunitinib-treated renal cell carcinoma subjects (Van der Veldt 2011), the 3 SNPs in NR1I3 (were analyzed separately and together as a haplotype for association with clinical endpoints (see Tables 1 and 2). Similarly, the 3 SNPs in ABCB1 (ATP-binding cassette, subfamily B, member 1) were also analyzed separately and together as a haplotype.

Because of the limited sample size of the study, genetic analyses were performed without adjustment for baseline demographics or potential covariates, and p-values were not adjusted for multiple testing.

### Pharmacogenetic Results:

### Genotyping Results:

A summary of the genotyping results for the 20 functional SNPs is included in Table 1. All the PGx consented subjects (n=31) were successfully genotyped. A Hardy-Weinberg equilibrium (HWE) analysis was performed to test if the distribution of the observed genotypes in foretinib treated Asian subjects deviated from that expected according to the Hardy-Weinberg principle (i.e., HWE p<0.05). No deviation from HWE was observed (Table 1).

TABLE 1
List of Single Nucleotide Polymorphisms Genotyped for PGx Analysis

| | | | | | | |
|---|---|---|---|---|---|---|
| | | | | Observed | Genotype | |
| | | | | MAF in PGx | Count in PGx | |
| | | | MAF in Asians | Population | Population | HWE |
| Gene | Polymorphism | rsID | (%)* | (%) | (aa/Aa/AA)** | (p-value) |

Tier 1: SNPs with Reported Evidence for Association with Efficacy of Kinase Inhibitors

| | | | | | | |
|---|---|---|---|---|---|---|
| VEGFA | −2578A/C; near 5′end | rs699947 | A = 0.24-0.37 | A = 0.23 | (3/8/19) | 0.16 |
| IL-8 | 2767A/T; 3′UTR | rs1126647 | T = 0.31-0.35 | T = 0.40 | (5/14/11) | 0.88 |
| IL-6 | −572 G/C; near 5′end | rs1800796 | G = 0.21-0.23 | G = 0.23 | (2/10/18) | 0.78 |
| | −6101A/T; near 5′end | rs4719714 | T = 0.06-0.19 | T = 0.25 | (1/13/16) | 0.48 |
| | −6331 T/C; near 5′end | rs10499563 | C = 0.06-0.21 | C = 0.25 | (1/13/16) | 0.78 |
| NR1I2/PXR | −25385C/T; 5′UTR | rs3814055 | T = 0.22-0.41 | T = 0.13 | (0/8/22) | 0.39 |
| NR1I3/CAR | 5719C/T; P180P | rs2307424 | C = 0.45-0.56 | C = 0.42 | (6/13/11) | 0.55 |
| | 7738A/C; intronic | rs2307418 | C = 0.06-0.11 | C = 0.12 | (0/7/23) | 0.47 |
| | 7837T/G; intronic | rs4073054 | G = 0.03-0.20 | G = 0.10 | (1/4/25) | 0.15 |

Tier 2: SNPs altering the activity of enzymes and transporters responsible for foretinib ADME

| | | | | | | |
|---|---|---|---|---|---|---|
| CYP3A4 | −392A/G; near 5′end | rs2740574 | G = 0.01 | G = 0.02 | (0/1/29) | 0.93 |
| ABCB1 (P-gp) | 1236T/C; G412G | rs1128503 | C = 0.29-0.41 | C = 0.30 | (5/8/17) | 0.05 |
| | 2677G/T/A; A893S/T | rs2032582 | G = 0.35-0.49 | G = 0.45 | (7/13/10) | 0.49 |
| | 3435C/T; I1145I | rs1045642 | C = 0.40-0.63 | C = 0.48 | (8/13/9) | 0.47 |
| ABCG2 (BCRP) | 421C/A; Q141K | rs2231142 | A = 0.06-0.34 | A = 0.28 | (4/9/17) | 0.15 |

Tier 3: SNPs altering the expression levels of cytokines/growth factors that are important
in HCC pathobiology or are targeted by foretinib

| | | | | | | |
|---|---|---|---|---|---|---|
| ANGPT2 | A/T; intronic | rs1868554 | T = 0.50-0.64 | T = 0.45 | (4/19/7) | 0.13 |
| C-MET | −312G/C; near 5′end | rs1858830 | C = 0.37 | C = 0.37 | (4/14/12) | 0.98 |
| HGF | −1652 C/T; near 5′end | rs3735520 | T = 0.38-0.52 | T = 0.38 | (5/13/12) | 0.65 |
| LEP (Leptin) | −2548 G/A; near 5′end | rs7799039 | G = 0.20-0.51 | G = 0.27 | (1/14/15) | 0.29 |
| PDGFRA | −573G/T; near 5′end | rs1800812 | T = 0.15-0.27 | T = 0.10 | (0/6/24) | 0.54 |
| IGF2 | A/G; intronic | rs3741208 | A = 0.17-0.28 | A = 0.15 | (2/5/23) | 0.06 |

Abbreviations: MAF = Minor Allele Frequency; HWE = Hardy-Weinberg Equilibrium; UTR = untranslated region; VEGFA = Vascular endothelial growth factor A; IL-8 = Interleukin-8; IL-6 = Interleukin-6; NR1I2/PXR = nuclear receptor subfamily 1, group I, member 2/Pregnane X Receptor; NR1I3/CAR = nuclear receptor subfamily 1, group I, member 3/Constitutive Androstane Receptor; CYP3A4 = cytochrome P450, subfamily IIIA, polypeptide 4; ABCB1 (P-gp) = ATP-binding cassette, subfamily B, member 1 (P-glycoprotein); ABCG2 (BCRP) = ATP-binding cassette, subfamily G, member 2 (Breast Cancer Resistance Protein); ANGPT2 = Angiopoietin 2; C-MET = MET protooncogene; HGF = Hepatocyte growth factor; LEP = Leptin; PDGFRA = Platelet derived growth factor receptor alpha; IGF2 = Insulin-like growth factor 2
*The Asian subjects enrolled in MET111645 were of East Asian, South East Asian or Central/South Asian origin. Since the minor allele frequencies (MAF) of SNPs vary among these different Asian populations, a range for MAFs is included in the table. (Source for Asian MAFs: 1000 Genomes and HapMap Populations).
**Genotypes: aa = homozygous minor, AA = homozygous major, Aa = heterozygous.

Results for Association of Genetic Variants with Clinical Endpoints:

Overall, no statistically significant associations were detected between ORR and any of the genetic variants or haplotypes evaluated.

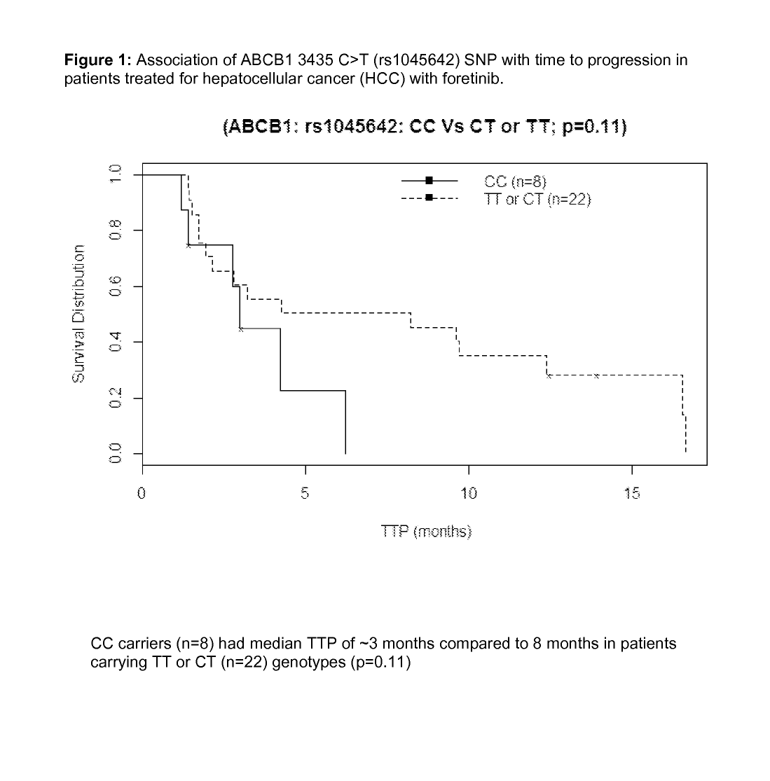

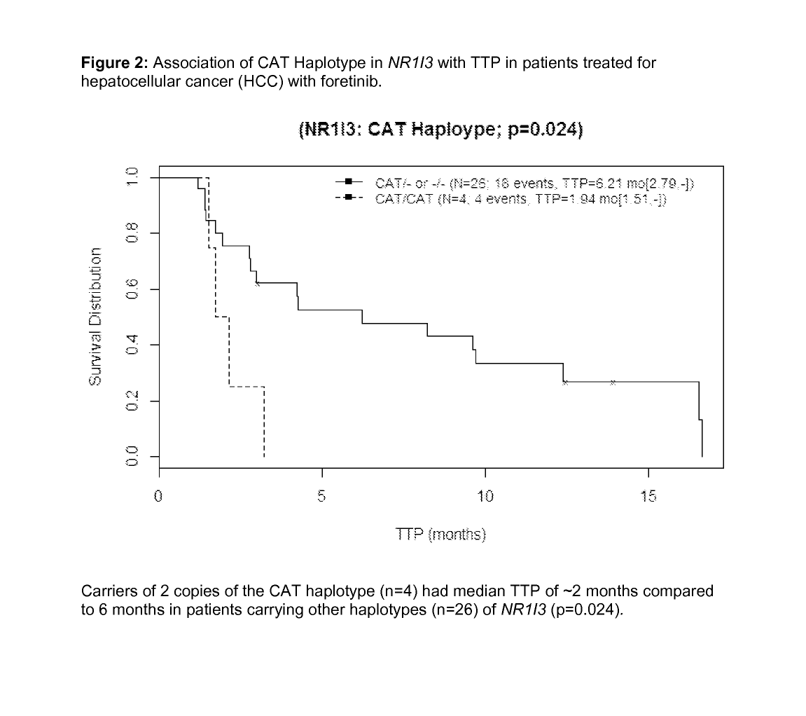

The CAT haplotype in NR1I3/CAR was significantly associated with TTP in foretinib treated HCC subjects. The presence of 2 copies of CAT in HCC subjects receiving foretinib was associated with inferior TTP (median TTP: ˜2 months) compared with patients with 1 or no copy (median TTP: ˜6 months; p=0.024, Table 2 and FIG. 2). Similarly, a synonymous SNP in ABCB1 (SNP ID: rs1045642; 3435 C>T, Ile114511e) also showed a suggestive trend for association with inferior TTP in foretinib treated HCC subjects (median TTP: 3 months (CC genotype) vs. 8 months (CT+TT); p=0.11, Table 2 and FIG. 1).

NR1I3/CAR (Constitutive Androstane Receptor), a nuclear receptor, regulates the expression of cytochrome P450, subfamily IIIA, polypeptide 4 (CYP3A4) and ABCB1, which code for proteins responsible for foretinib metabolism and efflux, respectively.

While the intronic SNP, rs2307418 (7738 A/C) present in the NR1I3 haplotype is reported to disrupt the binding site for transcription factor NF-1, the effect of the SNP or the haplotype on regulating CYP3A4 and ABCB1 activity and in turn, foretinib metabolism and efflux is not known.

On the other hand, the CC genotype of the ABCB1 synonymous SNP, rs1045642 (3435 C>T) has been reported to increase the mRNA/protein expression of MDR1/P-gp and improve its activity due to improved co-translational folding compared to TT genotype (Kimchi-Sarfaty, 2007). However, its effect on drug pharmacokinetics (PK) (e.g. digoxin) has been equivocal in a number of reports. It is possible that CC carriage may lead to better MDR1/P-gp efflux activity compared to CT or TT carriage, resulting in reduced accumulation of foretinib in hepatocytes and in turn poor treatment outcome in HCC.

None of the genetic variants or combinations of variants (haplotypes) was significantly associated with overall survival (OS) (p>0.05). The CAT haplotype in gene NR1I3 showed a suggestive trend for association with inferior OS (median OS: 5.9 months [CAT/CAT] vs 8.5 months [other haplotypes]; p=0.11).

TABLE 2
SNPs/Haplotypes with Moderate Statistical Correlation with TTP

| | | Genotype/ | Number of | Median TTP | |
|---|---|---|---|---|---|
| | SNP | Haplotype | Subjects | (months) | TTP (p-value) |
| NR1I3 | rs2307424, | CAT/CAT | 4 | 1.92 (1.51-NE*) | 0. 024 |
| Haplotype | rs2307418, | | | | [CAT/CAT vs. |
| | rs4073054 | | | | Others] |
| | | [CAT/− or −/−] | 26 | 6.21 (2.96-NE) | |
| ABCB1 | rs1045642 | CC | 9 | 2.96 (2.76-NE) | 0.11 (CC vs. TC + TT) |
| | | [TC or TT] | 21 | 8.21 (2.17-NE) | |

*NE = Not Evaluable (the 95% upper confidence boundary could not be accurately estimated from the regression model)

### Pharmacogenetic Analysis Conclusions:

This exploratory PGx analysis investigated the association between 20 genetic polymorphisms and foretinib response of Asian HCC subjects. While it is not possible to draw definitive conclusions because of the limited subject numbers and the exploratory nature of this PGx analysis, the data suggested that in advanced HCC subjects treated with foretinib, a haplotype of three germline SNPs in NR1I3 (CAT haplotype) and a germline SNP in ABCB1 may be associated with inferior TTP. It is hypothesized that there may be more rapid metabolism and/or efflux of foretinib in patients carrying the higher expression genotypes, resulting in reduced efficacy. Recognizing these suggestive associations are exploratory and determined in a small sample of HCC subjects, they are not actionable clinically and will require confirmation in an independent dataset.

### REFERENCES

Van der veldt et al. Genetic polymorphisms associated with a prolonged progression-free survival in patients with metastatic renal cell cancer treated with sunitinib. Clin Cancer Res. 2011 Feb. 1; 17(3):620-9. Epub 2010 Nov. 19.
Kimchi-Sarfaty et al. A “silent” polymorphism in the MDR1 gene changes substrate specificity. Science. 2007 Jan. 26; 315(5811):525-8. Epub 2006 Dec. 21.
Miyahara et al. Predicting the treatment effect of sorafenib using serum angiogenesis markers in patients with hepatocellular carcinoma. J Gastroenterol Hepatol. 2011 November; 26(11):1604-11. doi: 10.1111/j.1440-1746.2011.06887.x.
Harmon 2011 et al. Mechanism-related circulating proteins as biomarkers for clinical outcome in patients with unresectable hepatocellular carcinoma receiving sunitinib. J Transi Med. 2011 Jul. 25; 9:120.

### Example 2

### Circulating Pharmacodynamic (PD) Markers

Blood was collected at baseline (Day 1) and post treatment with 30 mg/daily of foretinib on Days 8, 15, and 22. CAF (Cytokine and Angiogenic Factor) levels were determined. CAF's evaluated are listed in Table 3. CAF levels were determined by using the Searchlight platform (Aushon Biosciences). Levels of circulating sMET and HGF were determined using Meso Scale Discovery Platform (Don Bottaro, National Cancer Institute [NCI]).

TABLE 3
CAF panel.

| CAF | Definition | Function |
|---|---|---|
| IL6 | Interleukin-6 | Cytokine |
| PLGF | Placental Growth Factor | Growth Factor |
| TM | Thrombomidulin | Invasion/matrix |
| TGFB1 | Transforming growth factor beta1 | Growth Factor |
| HGF | Hepatocyte growth factor | Growth Factor |
| FASL | Fas ligand (TNF ligand superfamily, | Apoptosis |
| | member 6) | |
| GCSF | Granulocyte colony-stimulating factor | Angiogenesis |
| IL8 | Interleukin-8 | Cytokine |
| TRAIL | TNF related apoptosis-inducing ligand | Apoptosis |
| ANG2 | Angiopoietin 2 | Angiogenesis |
| FGFb | Fibroblast growth factor 2 | Growth Factor |
| SCF | MGF stem cell factor | Angiogenesis |
| VEGF | Vascular endothelial growth factor | Angiogenesis |
| BMP9 | Bone morphogenetic protein 9 | Invasion/matrix |
| OPN | Osteopontin | Matrix |
| ECadherin | Caderin1 (CDH1) | EMT |
| EGF | Epidermal growth factor | Growth Factor |
| ESelectin | Selectin E | Adhesion/matrix |
| IGFBP1 | Insulin-like growth factor-binding | Adhesion/matrix |
| | protein 1 | |
| Leptin | Leptin | Invasion/matrix |
| TSP2 | Thrombospondin 2 | Invasion/matrix |
| VEGFR2 | VEGF receptor 2 | Angiogenesis |
| IGFBP3 | Insulin-like growth factor-binding | Invasion/ |
| | protein 3 | metastasis |
| MMP9 | Matrix metalloproteinase 9 | Invasion/ |
| | | metastasis |
| TIMP2 | Tissue inhibitor of | Invasion/ |
| | metalloproteinase −2 | metastasis |
| VCAM1 | Vascular cell adhesion molecule 1 | Adhesion/matrix |
| Clusterin | Clusterin | Apoptosis |
| Fibronectin | Fibronectin | Adhesion/matrix |

EMT = Epithelial-mesenchymal transition
TNF = Tumor necrosis factor

### Statistical Methods

### Biomarker Population.

The biomarker analysis population was predefined to include patients who met the following criteria:
Patient is in a 30 mg cohort.
Pharmacodynamic (PD)-sample data available at baseline and for at least 1 post-baseline timepoint, and
Patient received at least 75% of planned doses up to time of last PD sample.

### Covariates.

Where noted, multivariate analyses were conducted with the following covariates: eastern cooperative oncology group (ECOG) performance status (normal vs symptomatic), hepatitis infection (current vs none), cirrhosis (current vs none), Sex, Age, baseline tumor burden (as measured by sum of longest lesion diameters [SLD]).

### CAF Time Course.

CAF percent changes from baseline were assessed for each CAF, at each time point, for available data in the biomarker population, using the Wilcoxon Signed Rank Test.

### Correlation of Baseline (Day 1) CAF Levels and Baseline Tumor Burden.

Correlation of day 1 CAF levels and tumor burden, as measured by sum of longest lesion diameters, was assessed using the Spearman Rank Correlation test.

### Association Between Baseline (Day 1) CAF Levels and Clinical Response.

Association between day 1 CAF levels and clinical response (CR, PR, SD vs PD as determined by mRECIST) was assessed using univariate and multivariate (covariate adjusted) logistic regression analysis. CAF data were transformed (log₂) where appropriate.

### Association Between Baseline (Day 1) CAF Levels and Time to Progression (TTP).

Association between day 1 CAF levels and TTP was assessed using univariate and multivariate (covariate adjusted) proportional hazards regression analysis. CAF data were transformed (log₂) where appropriate.

### Association Between Baseline (Day 1) CAF Levels and Overall Survival (OS).

Association between day 1 CAF levels and OS was assessed using univariate and multivariate (covariate adjusted) proportional hazards regression analysis. CAF data were transformed (log₂) where appropriate. For statistically significant CAF's in the OS statistical models, a combined model of the significant CAF's will be explored, in which an optimal predictive combination of the CAF's (including optimal cutoffs) will be investigated. The method employed to explore the CAF's will depend upon the number of significant CAF's, and will be described in the results section.

All analyses were conducted using SAS v9.1.3; p<0.01 was considered statistically significant.

### Results

GCSF and Leptin had CAF levels below LOQ and replaced with ½ LOQ. For GCSF, 12% overall, and 8% (3/38) at baseline, were replaced. For Leptin, 11% overall, and 8% (3/38) at baseline, were replaced. CAF levels for FGFb were below the limit of quantitation (LOQ) for more than 80% of the samples, therefore no analyses were conducted for FGFb.

### Biomarker Population.

A total of 38 patients are included in the biomarker population.

### CAF Time Course.

Of the 28 CAF's and 2 NCI proteins, 15 showed statistically significant changes from baseline at one or more post-baseline time points (Table 4). Most of the changes were increases (11/15).

TABLE 4
CAF with statistically significant change from baseline (p < 0.01)

| CAF | Day | Percent Change | pValue |
|---|---|---|---|
| ANG2 | 15 | −24 | 0.0001 |
| ANG2 | 22 | −17 | 0.0055 |
| Clusterin | 8 | 25 | 0.0017 |
| Clusterin | 22 | 23 | 0.0003 |
| FASL | 8 | 26 | 0.0001 |
| FASL | 15 | 20 | 0.0001 |
| FASL | 22 | 44 | 0.0001 |
| GCSF | 8 | 46 | 0.0018 |
| IGFBP3 | 8 | 19 | 0.0022 |
| IGFBP3 | 15 | 13 | 0.0086 |
| IGFBP3 | 22 | 17 | 0.0084 |
| IL6 | 8 | 58 | 0.0001 |
| IL6 | 15 | 124 | 0.0001 |
| IL6 | 22 | 111 | 0.0001 |
| IL8 | 15 | 20 | 0.0086 |
| PLGF | 8 | 105 | 0.0001 |
| PLGF | 15 | 130 | 0.0001 |
| PLGF | 22 | 120 | 0.0001 |
| SCF | 15 | −14 | 0.0055 |
| TRAIL | 8 | 25 | 0.0001 |
| TRAIL | 15 | 17 | 0.0027 |
| TRAIL | 22 | 24 | 0.0013 |
| TSP2 | 8 | −14 | 0.004 |
| TSP2 | 15 | −19 | 0.0011 |
| VCAM1 | 8 | 28 | 0.0001 |
| VCAM1 | 15 | 43 | 0.0001 |
| VCAM1 | 22 | 64 | 0.0001 |
| VEGF | 8 | 61 | 0.0001 |
| VEGF | 15 | 83 | 0.0001 |
| VEGF | 22 | 83 | 0.0001 |
| VEGFR2 | 15 | −21 | 0.0001 |
| VEGFR2 | 22 | −21 | 0.0001 |
| sMET | 8 | 12 | 0.0001 |

### Correlation of Baseline (Day 1) CAF Levels and Baseline Tumor Burden.

5 CAF's had a statistically significant positive association with baseline tumor burden (Table 5). There were no significant negative associations.

TABLE 5
CAF with Statistically significant correlation of baseline (day 1)
levels and baseline tumor burden (SLD).

| CAF | Correlation | pValue | N |
|---|---|---|---|
| ANG2 | 0.62 | 0.0001 | 35 |
| IGFPB1 | 0.56 | 0.0005 | 35 |
| IL8 | 0.56 | 0.0005 | 35 |
| OPN | 0.48 | 0.0035 | 35 |
| TSP2 | 0.45 | 0.0064 | 35 |

### Association Between Baseline (Day 1) CAF Levels and Clinical Response.

No significant associations in univariate or multivariate models.

### Association Between Baseline (Day 1) CAF Levels and Time to Progression (TTP).

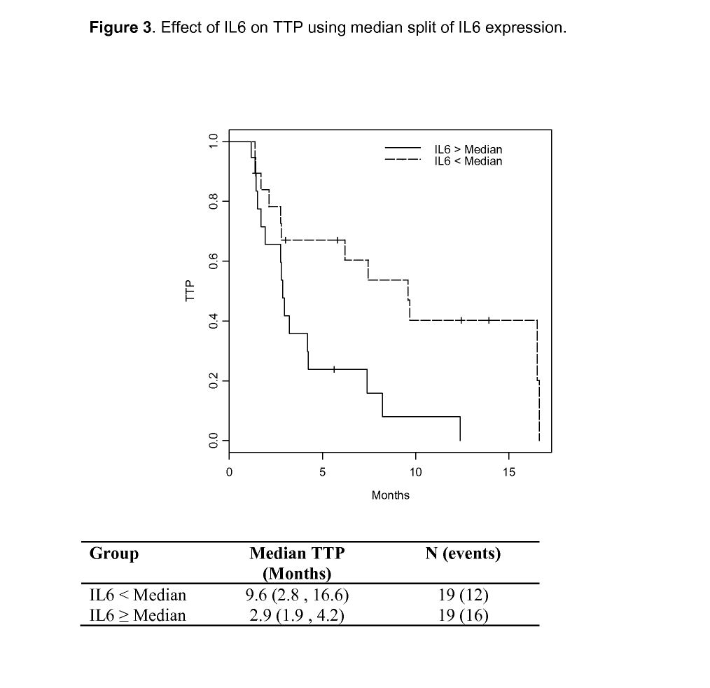

In univariate models, higher baseline levels of IL6 and MMP9 are associated with shorter time to progression (Table 6). In multivariate models, IL6 is an independent predictor of TTP. Using median split of IL6 levels, patients with lower levels of IL6 have ˜6.7 month longer TTP (9.6 months vs 2.9 months), compared to patients with higher levels of IL6 (FIG. 3).

TABLE 6
CAF with statistically significant association with TTP.

| CAF | pValue | Hazard Ratio (95% Cl)) |
|---|---|---|
| IL6 | 0.0024 | 1.44 (1.14, 1.82) |
| MMP9 | 0.0109 | 1.75 (1.14, 2.68) |

### Association Between Baseline (Day 1) CAF Levels and Overall Survival (OS).

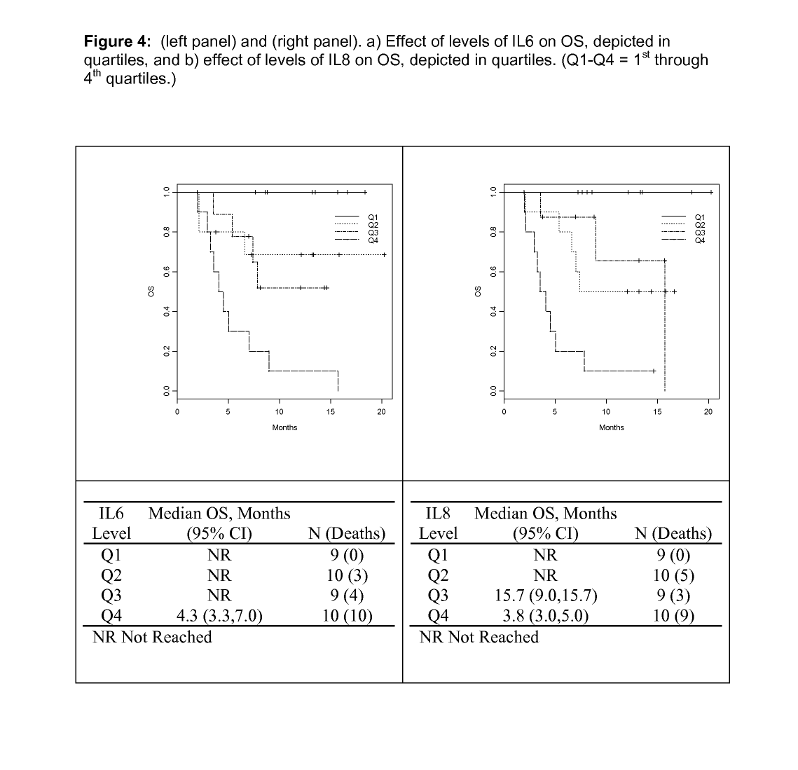

In univariate models, higher baseline levels of IL6, IL8, TSP2, MMP9, and IGFBP1 were associated with shorter OS times (Table 7). In multivariate models, IL6 and IL8 are independent predictors of OS. Individual effects of IL6 and IL8 on survival times are depicted by using quartile splits of the CAF levels for each of these markers (FIG. 4). The effect of levels of IL6 and IL8 are clear from FIG. 4: patients in the lowest quartiles have 0/9 deaths (all patients censored) while patients in the highest quartile have 10/10 (IL6) and 9/10 (IL8) deaths, respectively. Patients with intermediate levels (2^{nd} and 3^{rd} quartiles) have intermediate OS.

TABLE 7
CAF with statistically significant association with OS.

| CAF | P-value | Hazard Ratio (95% CI) |
|---|---|---|
| IL8 | <.0001 | 2.38 (1.62, 3.49) |
| IL6 | 0.0002 | 1.79 (1.31, 2.43) |
| TSP2 | 0.0024 | 2.21 (1.33, 3.70) |
| MMP9 | 0.0059 | 2.18 (1.25, 3.80 |
| IGFBP1 | 0.0071 | 1.48 (1.11, 1.96) |

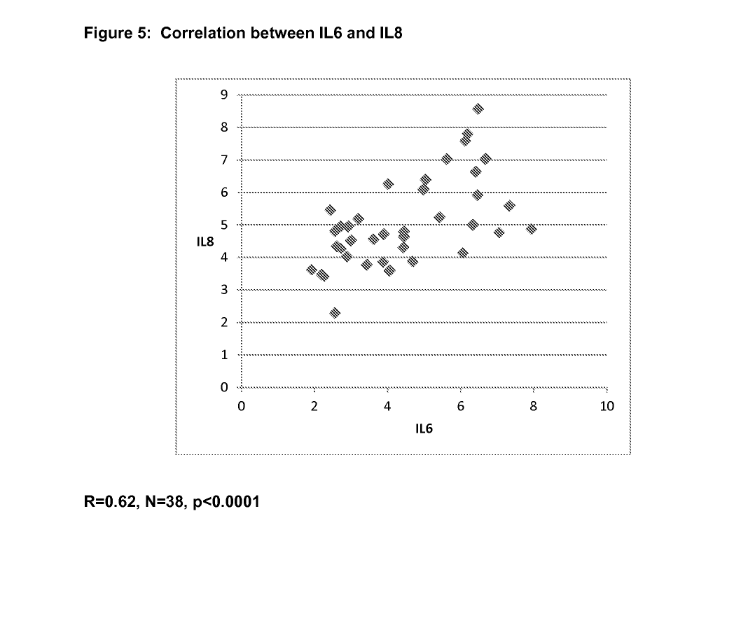

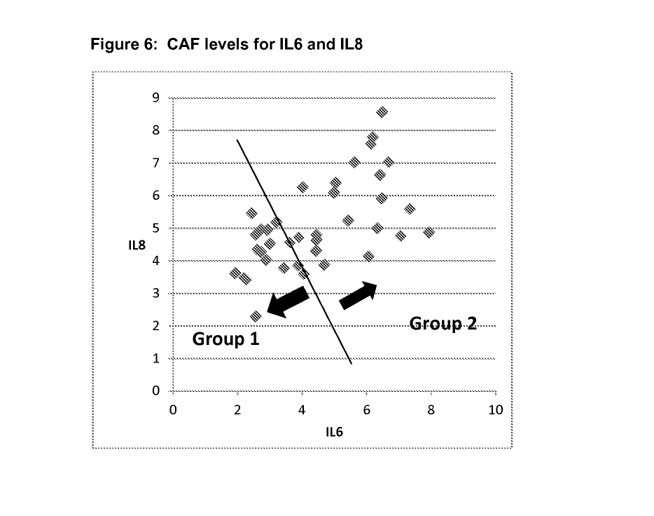

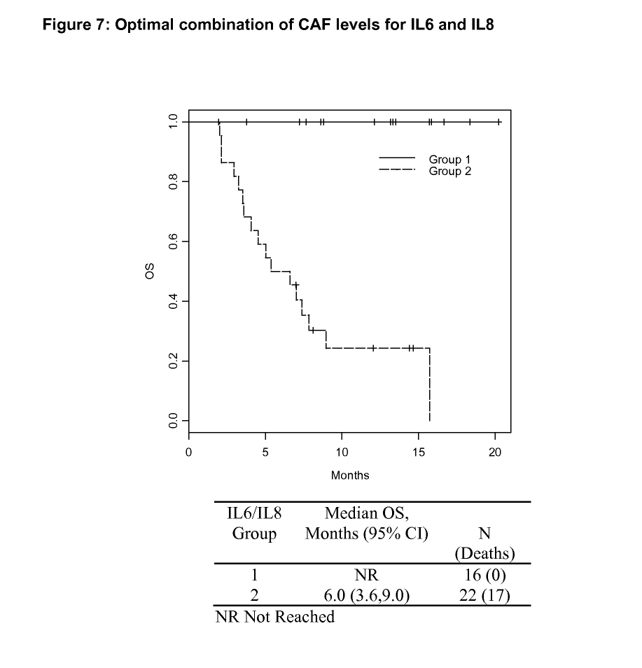

Levels of IL6 and IL8 were correlated (FIG. 5, Spearman's R=0.62, p<0.0001). Using a grid search approach, where all possible cutoffs of IL6 and IL8 were evaluated (subject to the constraint that at least 5 subjects were present above or below a cutoff), and searching for the lowest log rank p-value, the optimal combination of CAF levels for IL6 and IL8 were identified, which maximized OS. The optimal combination is illustrated (FIG. 7). Using Kaplan-Meier analysis, the combination of IL6 and IL8 which maximizes OS is a strong predictor of patient survival (FIG. 6). Patients with favorable levels of IL6 and IL8 (group 2 in FIG. 6) are all censored (N=16), whereas patients with unfavorable levels of IL6 and IL8 (group 1 in FIG. 6) have only 5 censored, and 17 deaths, with a median survival of 6 months. Clearly, levels of IL6 and IL8, alone and in combination, are strongly related to OS in this patient population.

### DISCUSSION

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A method of treating cancer in a patient in need thereof comprising:
determining whether said patient has less than 2 copies of the CAT haplotype in NR1I3 gene and/or TT or CT genotype at the rs1045642 reference single nucleotide polymorphism in ABCB1 gene; and
if said patient has less than 2 copies of the CAT haplotype in NR1I3 gene and/or TT or CT genotype at the rs1045642 reference single nucleotide polymorphism in ABCB1 gene, administering to said patient a c-Met inhibitor.

2. The method of claim 1 wherein the patient has been previously genotyped as having less than 2 copies of the CAT haplotype in NR1I3 gene and/or a genotype TT or CT genotype at the rs1045642 reference single nucleotide polymorphism in ABCB1 gene, comprising administering to the patient a c-Met inhibitor.

3. The method of claim 1, wherein said determining comprises testing said patient for presence of less than 2 copies of the CAT haplotype in NR1I3 gene and/or the TT or CT genotype at the rs1045642 reference single nucleotide polymorphism in ABCB1 gene.

4. The method of claim 1, wherein the patient has hepatocellular carcinoma.

5. The method of claim 1, wherein the patient has a cancer selected from the group consisting of: colon cancer, breast cancer, renal cell carcinoma, melanoma, lung cancer including non-small cell lung cancer and adenocarcinoma, gastric cancer, colorectal cancer, neuroendocrine cancer, thyroid cancer, head and neck cancer, brain cancer, cervical cancer, bladder cancer, esophageal cancer, pancreatic cancer, prostate cancer, mesothelioma, liver-hepatobiliary cancer, multiple myeloma, leukemia, thyroid cancer including Hurthle cell, muscle sarcoma (leiomyosarcoma), hepatocellular cancer and bone sarcoma (chonrosarcoma).

6. The method of claim 1, wherein said c-Met inhibitor is a compound of Formula (I): 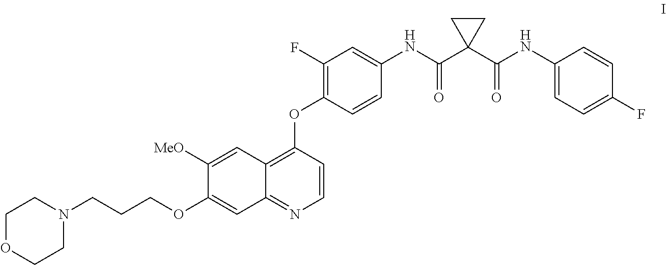 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt thereof.

7. The method according to claim 6, wherein said Formula I is the free base form.

8. The method of claim 1 wherein said patient is of Asian heritage.

9. The method of claim 1 wherein the c-Met inhibitor is administered at a dose of at least about 30 mg daily.

10. The method of claim 1 wherein said c-Met inhibitor is administered as monotherapy.

11. The method of claim 1 wherein said c-Met inhibitor is co-administered with at least one other anti-neoplastic agent.

12. A method of treating a human with cancer comprising:
determining whether said human has the TT or CT genotype at the rs1045642 reference single nucleotide polymorphism in ABCB1 gene; and/or
determining whether said human has less than 2 copies of the CAT haplotype in the NR1I3 gene and
if said human has the TT or CT genotype at the rs1045642 reference single nucleotide polymorphism in ABCB1 gene and/or has less than 2 copies of the CAT haplotype in the NR1I3 gene, administering to said human a c-Met inhibitor.

13. The method according to claim 12, wherein said c-Met inhibitor is a compound of formula (I): 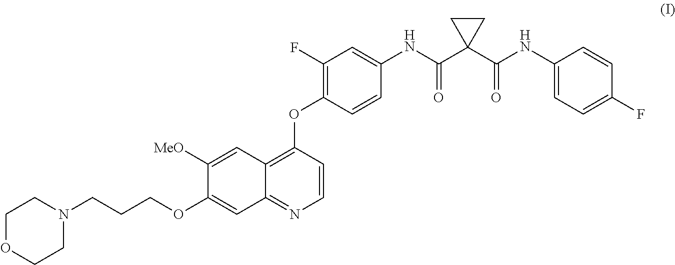 [SMILES: COc1cc2c(Oc3ccc(NC(=O)C4(C(=O)Nc5ccc(F)cc5)CC4)cc3F)ccnc2cc1OCCCN1CCOCC1]
or a pharmaceutically acceptable salt or solvate thereof.

14. The method of claim 12 wherein said cancer is hepatocellular carcinoma.

15. A method of treating a patient having heptocellular carcinoma comprising determining whether a said patient has an elevated level of at least one cytokine or angiogenesis factor (CAF) selected from: IL8, IL6, TSP2, MMP9, and IGFBP1 and if said human does not have and elevated level of at least one of said CAF administering a therapeutically effective amount of a c-Met inhibitor to said patient.

16. A method of treating cancer in a patient in need thereof comprising:
administering to the patient a c-Met inhibitor; and then
determining whether said patient has less than 2 copies of the CAT haplotype in NR1I3 gene and/or the TT or CT genotype at the rs1045642 reference single nucleotide polymorphism in ABCB1 gene.
